(19) United States
(12) Patent Application Publication
(10) Pub. No.: US 2004/0029229 A1
(43) Pub. Date: Feb. 12, 2004

(54) High level protein expression system

(76) Inventors: Philip J. Reeves, Somerville, MA (US); H. Gobind Khorana, Belmont, MA (US); Nico Callewaert, Lichtervelde (BE); Roland Contreras, Merelbeke (BE)

Correspondence Address: NIXON PEABODY LLP, ATTENTION: DAVID RESNICK, 101 FEDERAL STREET, BOSTON, MA, 02110, US

(21) Appl. No.: 10/441,885

(22) Filed: May 20, 2003

Related U.S. Application Data
(60) Provisional application No. 60/381,978, filed on May 20, 2002.

(51) Int. Cl. C12N 5/08; C12P 21/02

(52) U.S. Cl. 435/69.1; 435/369; 435/320.1

(57) ABSTRACT

The present application is directed to a system for expressing high levels of a protein of interest in a cell. Preferably the system includes introducing into a mammalian cell line an expression vector that comprises a gene encoding a selectable marker under the control of a weak promoter to facilitate integration of the expression vector into genomic areas that result in high levels of expression, along with a gene encoding the protein of interest. The gene of interest is linked to a strong promoter which results in high levels of expression of the desired protein. In one embodiment, the protein of interest is detrimental or toxic to cells, and the promoter is an inducible promoter. In another embodiment, the protein of interest is a glycoprotein, including membrane proteins, and the cell line has been mutated to inactivate N-acetylglucosamine transferase I to ensure uniform glycosylation. Preferably the cell is a mammalian or an insect cell.

### CROSS-REFERENCE TO RELATED APPLICATIONS

This application claims the benefit of U.S. Provisional Application 60/381,978, filed May 20, 2002.

This invention was supported by National Institutes of Health grant GM28289 and the government of the United States has certain rights thereto.

### FIELD OF THE INVENTION

The present application is directed to a system for expressing high levels of a protein of interest in a cell. Preferably the system includes introducing into a mammalian cell line an expression vector that comprises a gene encoding a selectable marker under the control of a weak promoter to facilitate integration of the expression vector into genomic areas that result in high levels of expression, along with a gene encoding the protein of interest. The gene of interest is linked to a strong promoter which results in high levels of expression of the desired protein. In one embodiment, the protein of interest is detrimental or toxic to cells, and the promoter is an inducible promoter. In another embodiment, the protein of interest is a glycoprotein, such as a membrane protein, and the cell line has been mutated to inactivate N-acetylglucosamine transferase I to ensure uniform glycosylation. Preferably the cell is a mammalian or an insect cell.

### BACKGROUND OF THE INVENTION

With the advent of molecular biology and recent advances in genomics, the ability to clone and express recombinant proteins in large amounts has become increasingly important. The ability to purify high levels of proteins is important in the human pharmaceutics and biotechnology setting, for production of protein pharmaceuticals such as insulin, as well as in the basic research setting, for example to crystallize a protein to allow determination of its three-dimensional structure. Proteins that are otherwise difficult to obtain in quantity can be “overexpressed” in a host cell and subsequently isolated and purified. Preinsulin for example may be produced in a recombinant prokaryotic microorganism carrying DNA encoding rat preinsulin (U.S. Pat. Nos. 4,431,740 and 4,652,525).

Bacterial expression systems have been one approach to expression and purification of recombinant proteins. However, expression of many eukaryotic polypeptides, and particularly mammalian proteins, in bacterial cells has frequently produced disappointing and unsatisfactory results because conditions and the environment in the host cells were not conducive to correct folding and modification of the eukaryotic protein. For example, the formation of disulfide bonds is essential for the correct folding and stability of numerous eukaryotic proteins of importance to the pharmaceutical and bioprocessing industries. However, numerous studies over the last fifteen years have demonstrated that, with few exceptions, multidisulfide proteins cannot be expressed in active form in bacteria. Similarly, many eukaryotic proteins, including membrane and secreted proteins, have post-translational modifications including glycosylation, which play critical roles in both their correct folding and assembly as well as their function. However, bacteria lack the enzymes to add such modifications.

Yeast expression systems offer certain advantages for the production of some eukaryotic proteins, because they have powerful secretory pathways and have the ability to perform some limited post-translational modifications. However, yeast systems typically lead to improper folding of disulphide linked proteins, and may result in hypoglycosylation.

The use of mammalian cells for the production of eukaryotic proteins offers the important advantages of providing correct protein folding as well as the appropriate post-translational modifications, including glycosylation. Most of the soluble and membrane-bound proteins that are made in the endoplasmic reticulum, including those destined for transport to the Golgi apparatus, lysosomes, plasma membrane, or extracellular space are glycoproteins. In contrast, very few proteins in the cytosol are glycosylated, and those that are carry a much simpler sugar modification in which a single N-acetylglucosamine group is added to a serine or threonine residue of the protein. An extensive oligosaccharide trimming process takes place in the Golgi apparatus and determines the final glycosylation pattern on the mature protein.

Enzymatic protein glycosylation involves an initiation stage in which glycosyltransferases catalyze the addition of a monosaccharide, or in the case of asparagine N-linked glycosylation, a preformed oligosaccharide, to an amino acid residue in a given protein. The initiation step of protein glycosylation may be considered the key controlling event leading to the formation of a given glycopeptide linkage and involves the essential recognition events between the protein and glycosyltransferase, which determines the specific sites of glycan attachment. Processing of glycan chains involves the cooperative action of part of the estimated hundreds of different glycosyltransferases, successively adding a monosaccharide to the growing glycan chain. Identification and characterization of glycan structures of glycoproteins as well as the specific sites of glycan attachment are important for understanding the structure of a given glycoprotein, its function, and its immunobiology.

Although mammalian cells are uniquely equipped to process glycoproteins, a major disadvantage of mammalian protein expression systems is that yields of proteins are typically low. In addition, the expression of certain proteins or mutants may be toxic to the cell or otherwise detrimental to a cell, particularly when expressed in large quantities making it impossible to isolate a stable cell line that produces large levels of the protein.

In addition to the production of high levels of recombinant proteins for use as biopharmaceuticals, high levels of purified homogenous protein are essential for crystallization of membrane proteins for structure-function studies, including X-ray diffraction. The challenges of high levels of protein purification are demonstrated by structure-function studies of mammalian membrane proteins, including rhodopsin, which are often restricted by the limited amounts of purified material available.

Rhodopsin is the prototypical member of the family of seven-transmembranesegment, G protein-coupled receptors (GPCRs). GPCRs represent approximately 1-2 percent of the total proteins encoded by the human genome and are important targets for pharmaceutical intervention. For example, GPCRs include chemokine receptors such as CCR5 and CXCR4, which have been identified as cofactors in permitting the human immunodeficiency virus (HIV) to enter cells. Generally low levels of expression and the dependence of the native conformation of GPCRs on the hydrophobic, intramembrane environment have complicated the study of these proteins. Because of the difficulty in purifying large amounts of protein, the analysis of ligand interactions with GPCRs and screening for inhibitors of such interactions are commonly conducted using live cells or intact cell membranes. A significant drawback of such assays are the extremely large number of cells required for high-throughput screening. Furthermore, such studies can be complicated by the presence of numerous cell surface proteins, many of which are expressed at much higher levels than the GPCR of interest. Thus, certain approaches, such as using the GPCR-expressing cells to identify either natural or synthetic ligands in a complex mixture, are precluded. In addition, the generation of monospecific antibodies directed towards a particular GPCR in the complex cell membrane environment is inefficient. Furthermore, for some GPCRs, like the chemokine receptors, multiple ligands bind a single receptor, and conversely, a single ligand can bind multiple receptors. Therefore, if the cell expresses more than one receptor for the ligand being studied, interpretation of the results can be complicated.

Certain studies on rhodopsin and its mutants require relatively large amounts of materials. To meet these needs, stable mammalian cell line systems for high level expression of the opsin gene and its mutants were developed (1, 2). One challenge of mammalian expression systems is that expression of the recombinant gene may be toxic or otherwise detrimental to the cell, making it impossible to isolate stable cell lines expressing that gene. For example, a mammalian stable cell line expression system (1,2) for the production of milligram levels of rhodopsin mutants for NMR studies was recently reported (4-6). However, difficulties were encountered in constructing stable cell lines expressing adequate amounts of certain rhodopsin mutants and for constructing a stable cell line expressing a gene encoding rhodopsin kinase (7). For one rhodopsin mutant of particular interest (E113Q/E134Q/M257Y) stable cell lines could not be isolated after transfection of HEK293S cells with a plasmid constutively expressing this mutant gene. This mutant opsin exhibits strong (constitutive) activity in the non-retinal bound form which likely imparts toxicity.

One solution to overcome the difficulty of expressing a lethal protein is to use a strategy frequently used in bacterial expression systems (8,9) in which membrane proteins toxic to the bacterial cells are expressed by induction of the desired gene after achieving growth of cells to near maximum density. One example of an inducible gene expression system available in mammalian cells is a tetracycline (tet) regulated gene expression system, which was first described by Gossam and Bujard (11). By fusing the bacterial tet repressor (TetR) with the activating domain of the viral protein 16 (VP-16) a tetracycline controlled transactivator was generated that stimulated transcription of a desired gene from a minimal CMV promoter containing tet operator (tetO) sequences. Recently, by using the same tet repressor and operator components, Yao and Erikson have devised a tetracycline-inducible expression system that uses a full length CMV promoter (12). However, this strategy has only been used successfully for large-scale production of some but not all opsin mutants (10).

Another important use of high levels of purified rhodopsin is for crystallization of the protein to allow further structure-function studies. Crystallization of membrane proteins for X-ray diffraction structure determination is largely limited by availability of adequate amounts of purified homogeneous protein. Recently, X-ray diffraction quality crystals were grown using rhodopsin purified by extraction, using the detergent nonylglucoside, from sucrose density gradient refined ROS. While this work is an important advance for determination of the three dimensional structure of dark state wild-type rhodopsin, the purification method is clearly not suitable for preparation of rhodopsin mutants or for preparation of other G-protein coupled receptors that are naturally abundant in only limited quantities, and may be toxic to the cell.

For this reason, an expression system for rhodopsin has been developed that is based on the HEK293S cell line, a mammalian stable cell lines suitable for high level recombinant rhodopsin expression. Rhodopsin prepared using mammalian cell lines (including HEK293S cells) is correctly folded and functionally indistinguishable from bovine ROS rhodopsin. The HEK293S expression system was used recently for preparation of milligram amounts (10-40 mg) of labeled rhodopsin or rhodopsin mutants that was satisfactory for use both in solution NMR and magic angle spinning solid state NMR experiments. However, the N-glycans present on rhodopsin and other recombinant glycoproteins proteins expressed in HEK293S stable cell lines are extremely heterogeneous and can cause problems in crystallization.

One approach to deal with heterogenous N-glycans on a purified glycoprotein is to use tunicamycin treatment to eliminate all glycosylation. Thus, tunicamycin treatment along with a tetracycline-inducible expression has been used for purification of milligram quantities of non-glycosylated rhodopsin. However, this approach is not ideal because removing the N-glycans does not allow their role in the structure and function of the glycoprotein to be addressed. For example, although the precise role of glycosylation in rhodopsin structure and function is not fully understood, it clearly has an important role. Significant defects in signal transduction properties arising from the absence of glycosylation of the photoreceptor have been previously reported. Also, a rhodopsin mutant with three amino acid changes (E113Q/E134Q/M257Y) could not be purified when expressed in the presence of tunicamycin.

An alternative approach to deal with heterogenous N-glycans is to produce the protein in a cell which is defective in one of the various enzymes involved in N-glycan synthesis, such as GlcNAc transferase I. This approach has been used previously for isolation of a diverse collection of Chinese Hamster Ovary (CHO) cell lines resistant to various lectins resulting from deficiencies in various enzymes involved in N-glycan synthesis. However, it would be desirable to be able to use a wider range of cells for expression of proteins, particularly glycoproteins.

Accordingly, it would be desirable to have a method to express high levels of proteins which are properly folded with the appropriate post-translational modifications in high levels. It would also be desirable to have a method to express high levels of glycoproteins with homogenous modifications, including membrane proteins.

### SUMMARY OF THE INVENTION

The present invention provides a system for expressing high levels of a protein of interest in a cell. Preferably the system includes introducing into a mammalian cell line an expression vector that comprises a gene encoding a selectable marker under the control of a weak promoter to facilitate integration of the expression vector into genomic areas that result in high levels of expression, along with a gene encoding the protein of interest. The gene of interest is linked to a promoter which is efficient for high levels of expression of the desired protein.

Preferably, the promoter linked to the gene of interest is an inducible promoter. There are a wide range of inducible promoters. For example, the tet regulated gene expression system is preferred, in which a promoter such as the CMV promoter contains tet operator (tet O) sequences, and the host cells express tet repressor (tetR). Other inducible promoters can be obtained commercially from Ariad (Mass).

Another preferred embodiment provides that the weak promoter is a repressible promoter or an inducible promoter when the host cells are grown under non-inducing conditions. One preferred weak promoter is the H₂L^{d} promoter.

In one preferred embodiment of the invention, the expression plasmid contains the neomycin resistance gene under control of the weak H₂L^{d} promoter.

In one preferred embodiment, expression of the gene of interest in a host cell under protein-production conditions is detrimental to the host cell. For example, the gene of interest can operably linked to an inducible promoter, and the protein-production conditions comprise inducing conditions for the inducible promoter, and the protein expressed by the gene of interest is detrimental to the host cell. Alternatively, the protein-production conditions comprise addition of an agent which is detrimental to the host cell. One preferred class of agents are agents which inhibit the glycosylation pathway. Preferably, the agent which inhibits the glycosylation pathway is tunicamycin.

Preferred genes of interest encode proteins including glycoproteins, toxic gene products, membrane proteins, humanized proteins, antibodies, G-protein coupled receptors, and kinases. In another preferred embodiment the protein of interest is a membrane protein. Preferably, a seven-transmembrane spanning G protein coupled receptor. Particularly preferred proteins include opsin and rhodopsin.

Preferably, the selectable marker is encoded by neo, dhfr, puro, tk, or MDR.

In one particularly preferred embodiment, the gene of interest encodes a glycoprotein, and the host cell line generates uniform glycosylation pattern on glycoproteins. Preferably, the host cell line is HEK293. One further preferred embodiment provides a ricin resistant cell line that is defective in GnTi activity.

The present invention also provides methods of producing a protein of interest in a host cell line, comprising introducing a vector system into a host cell line, wherein the vector system comprises a selectable marker operably linked to a weak promoter and a gene of interest operably linked to a strong promoter; selecting stable transformants of said host cell line by isolating those transformants which express the selectable marker at a sufficient level to allow their survival when grown in the presence of a selecting agent; and culturing the transformants under growth conditions which allow expression of the gene of interest to produce the protein of interest.

In one preferred embodiment, the growth conditions comprise addition of a histone deacetylase inhibitor to the culture medium to increase expression levels of the gene of interest. Preferably, the histone deacetylase inhibitor is sodium butyrate, arginine butyrate, butyric acid, trapoxin, or trichostatin A. Even more preferably, sodium butyrate.

The present invention provides a system for high levels of expression of a protein of interest. Preferably, the protein of interest is expressed at a high level such that it can be purified from the host cell line at a quantity of at least 1 milligram per liter. More preferably, at least 5 milligrams per liter. Even more preferably, at least 10 milligrams per liter.

The present invention also provides methods for producing high levels of a protein of interest where the protein itself is detrimental to the cell, or the protein is produced under conditions which are detrimental to the cell. In one embodiment, the protein-production conditions comprise addition of an agent which is detrimental to the host cell. One preferred class of such agents are agents which inhibits the glycosylation pathway. Preferably, the glycosylation inhibitor is tunicamycin.

Preferably the host cell line is a mammalian or an insect cell. Particularly preferred mammalian cells include the HEK293S cell line.

One preferred embodiment provides high levels of expression of a glycoprotein. In this embodiment, the cell line has been mutated to generate uniform glycosylation patterns. For example, the cell line can be mutated to inactivate N-acetylglucosamine transferase I to ensure uniform N-glycans.

The present invention also provides a vector system for high level protein expression, wherein the vector system comprises a selectable marker operably linked to a weak promoter and a gene of interest operably linked to a strong promoter.

### BRIEF DESCRIPTION OF THE DRAWINGS

FIG. 1 depicts the vector for tetracycline-inducible opsin expression. pACMV[tetO] was constructed as described below. First, an oligonucleotide cassette containing two tetO operator sequences was inserted into a SacI site located near the 3′ end of the CMV promoter of plasmid pCEP4 (FIG. 1A). This CMV[tetO] promoter was isolated on a SalI and BamHI fragment (Fragment 1) and used to replace a KpnI-SalI DNA fragment in pACHEnc (FIG. 1B) that contained both a CMV promoter and a mutant acetylcholinesterase gene (16). The resulting plasmid (FIG. 1C) contains the CMV[tetO] promoter and a multiple cloning site as indicated. The opsin genes for expression were recovered from transient expression plasmids (pMT4) and inserted into the multiple cloning site as indicated. The resulting plasmid construct (FIG. 1D) was used to transfect HEK293S-TR cells. Stable cell lines were selected that were resistant to high concentrations of geneticin conferred by the neo gene, the expression of which is controlled by the weak H₂L^{d} promoter. Selected restriction enzyme sites are shown (italics) and those converted to blunt ends are underlined. The BamHI site was restored during construction of pACMV-tetO.

FIG. 2 is a graph showing expression of rhodopsin after growth of a stable cell line with or without induction of expression of the opsin gene. Cells were grown in dishes to near confluence and then incubated for the time period shown after addition of fresh growth medium supplemented as indicated. Each bar shows the average amount of rhodopsin produced by cells from duplicate culture dishes as determined by UV-vis difference spectroscopy (methods).

FIG. 3 shows the growth of a WT opsin inducible cell line in 1.1L suspension culture using a bioreactor. The cell line was grown in the bioreactor as described below. In FIG. 3A, the viable count was calculated by counting cells samples removed from the bioreactor at 24 hour intervals. The culture was fed supplemented (Sup.) and opsin expression was induced (Ind) as indicated by arrows and as described below. In FIG. 3B, samples collected throughout growth were solubilized and proteins were separated by SDS-PAGE (10%). Whole-cell proteins were detected by Coomassie blue staining. Proteins were transferred from gels to nitrocellulose by electroblotting and opsin was visualized by immundetection using anti-rhodopsin monoclonal antibody rho-1D4.

FIG. 4 shows the effect of tunicamycin concentration on the extent of rhodopsin N-glycosylation. The tetracycline inducible cell line producing wild type opsin was grown to near confluence in 10 cm diameter cell culture dishes. Spent medium was removed and replaced with fresh medium containing different concentrations (0-2.5-μg/ml) of tunicamycin. Three hours later expression of opsin was induced by further supplementation of the growth medium with tetracycline and sodium butyrate. Cells were harvest 48 hours later and treated with 11-cis retinal to constitute the rhodopsin pigment. Rhodopsin was purified and examined (0.5 μg) by SDS-PAGE using silver stain for detection. Rhodopsin purified from bovine ROS was loaded in lane 2 whereas molecular weight protein standards (M) were applied to lanes 1 and 8.

FIG. 5 shows the purification of non-glycosylated rhodopsin purified from a portion of the cells grown in a 5.5L bioreactor by rho-1D4-immunoaffinity chromatography. Elution buffers were E1, buffer H; E2, buffer I; E3, buffer J; E4, buffer K. Absorption at 280 nm (closed circles) and 500 nm (open circles) was recorded as indicated. (Inset) SD S-PAGE (10% gel) examination of selected fractions eluted from the column as visualized by silver stain and immunoblot after electroblotting to nitrocellulose. M, molecular weight standards, S, solubilized cell extract, F, Flow through (proteins that do not bind to rho-1D4-Separose).

FIG. 6 shows purification by rho-1D4-immunoaffinity chromatography of rhodopsin mutant (E113Q/E134Q/M257Y) from a portion of the cells grown in a 1.1L bioreactor culture. Elution buffers were E1, buffer B; E2, buffer C; E3, buffer D; E4, buffer E. Absorption at 280 nm (closed circles) and 380 nm (open circles) was recorded as indicated. (Inset) SDS-PAGE (10% gel) examination of selected fractions eluted from the column as visualized by silver stain and immunoblot after electroblotting to nitrocellulose. M, molecular weight standards, S, solubilized cell extract, F, Flow through (proteins that do not bind to rho-1D4-Separose).

FIG. 7 shows characterization of rhodopsin and its N-glycans prepared from ricin^{R} HEK293S cell lines. In FIG. 7A, HEK293S ricin^{R} cell lines expressing the opsin gene by transient transfection were used for purification of rhodopsin. Rhodopsin was characterized by SDS/PAGE (10%) followed by visualization of proteins by silver stain. Lanes 1 and 10 (M) (molecular weight standards); lanes 2-6, rhodopsin purified from HEK293S ricin^{R} cell lines obtained by using 1 ng/ml ricin; lane 7, rhodopsin purified from a ricin cell line obtained by using 10 ng/ml ricin; lane 8, rhodopsin from WT HEK293S cells; lane 9, rhodopsin from bovine ROS. In FIG. 7B, rhodopsin was prepared as described above. N-glycans removed by treatment with PNGaseF were analyzed as described below (see Example 2). Profile 1, profile of molecular weight standard (malto-oligosaccharides) and the corresponding number of glucose units indicated above the profile. N-glycan profiles of rhodopsin purified from HEK293S (profile 2), one HEK293S ricin^{R} cell line at 1 ng/ml ricin concentration (profile 3), the HEK293S ricin^{R} cell line at 10 ng/ml ricin concentration (profile 4), and bovine ROS (profile 5). The profile of RNase B N-glycans is in profile 6; the peak corresponding to Man₅GlcNAc₂ is indicated by an arrow. The peaks show relative fluorescence intensity corresponding to eluting N-glycans.

FIG. 8 shows assays of GnTI enzyme activity in the cell extracts. Triton X-100-solubilized cell extract were prepared from HEK293S WT or the HEK293S GnTI⁻ (ricin^{R}) cell line obtained by using 10 ng/ml ricin. The reaction mixtures contained 23.7 μg of HEK293S WT protein, 25.8 μg of HEK293S GnTI⁻ (ricin^{R}) protein, or 24.7 μg of protein of a mixture (1:1) of each lysate. GnTI enzymatic activity was measured by monitoring transfer of GlcNAc from UDP-GlcNAc to a fluorescently tagged acceptor (Man₅GlcNAc₂-APTS). Time-dependent formation of the product (GlcNAcMan₅GlcNAc₂-APTS) was detected by analysis of samples by PAGE by using an ABI377 sequencer system. Samples analyzed were prepared from HEK293S (circles), HEK293S GnTI⁻ (ricin^{R}) (triangles), or a 1:1 mixture of each lysate (squares).

FIG. 9 shows characterization of rhodopsin and rhodopsin N-glycans prepared from HEK293S and HEK293S-GnTI⁻ stable cell lines. The cell lines were induced for 2 days by using tetracycline and sodium butyrate. In FIG. 9A, rhodopsin was purified and examined by SDS/PAGE (10%) followed by silver stain. Lane 1, M (molecular weight standards); lane 2, rhodopsin from inducible HEK293S; lane 3, rhodopsin from HEK293S GnTI⁻, and lane 4, rhodopsin from ROS. FIG. 9B shows characterization of rhodopsin N-glycan composition. Profile 1, mobilities of molecular weight standards (malto-oligosaccharides) used for calibration. Profiles 2 and 3, N-glycans from rhodopsin samples prepared from inducible HEK293S cell line (before and after treatment with sialidase, respectively); profile 4, rhodopsin from HEK293S GnTI⁻; and profile 5, ROS rhodopsin.

FIG. 10 shows monitoring of opsin N-glycan formation and processing by pulse-chase. FIG. 10A shows analysis of opsin N-glycan formation in an inducible HEK293S cell line. Opsin expression was induced, the total mixture pulsed with [³⁵S]methionine for 40 min, and chased with cold methionine for times indicated. ³⁵S-methionine-labeled opsin was purified from DM-solubilized cell extracts by using rho1D4-Sepharose. Samples were examined by SDS/PAGE (lanes 2-9) and gels were dried before autoradiography. ¹⁴C-labeled markers (M) are in lanes 1 and 10. In FIG. 10B, the protocol was the same as in FIG. 10A, except that the HEK293S GnTI⁻ cell line was used.

FIG. 11 shows the growth and characterization of rhodopsin from inducible HEK293S GnTI⁻ cell line prepared in 1.1-liter suspension culture by using a bioreactor. The cell line was grown in suspension culture in a bioreactor. FIG. 11A shows viable cell count obtained by microscopic examination of samples removed from the bioreactor at 24-h intervals. The culture was supplemented (Sup.) and opsin expression was induced (Ind.) as indicated by arrows and described in Materials and Methods in ref. 26. In FIG. 11B, samples of cells collected throughout growth of the culture were solubilized buffer B, and the proteins were separated by SDS/PAGE (10%) and visualized by silver stain. Proteins were transferred from gels to nitrocellulose by electroblotting and opsin was visualized by immunoblotting with anti-rhodopsin mAb rho-1D4. The protein band corresponding to rhodopsin is indicated by the arrow. Lanes 1-8 are samples from days 1-8.

FIG. 12 shows purification of WT rhodopsin containing only the Man₅GlcNAc₂ N-glycan. HEK293S GnTI⁻ cells were collected from the bioreactor after induction of the opsin gene for 2 days (FIG. 11). A portion of the cells (one-third) were resuspended in buffer A and treated with 11-cis-retinal. For solubilization, nonylglucoside was added to produce the composition of buffer G. Elution buffers were E1, buffer H; E2, buffer I; E3, buffer J; E4, buffer K. In FIG. 12A, absorption at 280 nm (closed circles) and 500 nm (open circles) was recorded as indicated. (Inset) FIG. 12B shows SDS/PAGE (10% gel) examination of selected fractions eluted from the column as visualized by silver stain and immunoblot after electroblotting to nitrocellulose. M, molecular weight standards; S, solubilized cell extract; F, flowthrough (proteins that do not bind to rho-1D4-Sepharose). Migration of rhodopsin (rho) is indicated by an arrow.

FIG. 13 shows purification of rhodopsin mutant E113Q/E134Q/M257Y from the HEK293S GnTI stable cell line. FIG. 13A shows the column profile, showing A₂₈₀ and A₅₀₀. Fractions (1.5 ml) were collected and washes were E1 (buffer C), E2 (buffer D, pH 7.2), E3 as E2+100 μM 9-mer rhodopsin elution peptide, and E4 as E1+100 μM elution peptide. (Inset) FIG. 13B shows SDS/PAGE (10%) analysis of selected column fractions with detection either by silver stain or by immunoaffinity with rho-1D4. S, solubilized cell lysate; F, column flowthrough; M, molecular weight markers.

### DETAILED DESCRIPTION OF THE INVENTION

We have now discovered a system for expressing high levels of a protein of interest in a cell. Preferably the system includes introducing into a mammalian or insect cell line an expression vector that comprises a gene encoding a selectable marker under the control of a weak promoter to facilitate integration of the expression vector into genomic areas that result in high levels of expression, along with a gene encoding the protein of interest. The gene of interest is linked to a strong promoter which results in high levels of expression of the desired protein. Preferably, the promoter is an inducible promoter. Preferably, the protein of interest is a glycoprotein and the cell line has been mutated to inactivate N-acetylglucosamine transferase I to ensure uniform glycosylation. Preferably the protein of interest is a membrane protein. Preferably the cell is a mammalian or an insect cell. More preferably, the cell is a mammalian cell.

One aspect of the invention used to achieve high levels of expression of the gene of interest is the use of a weak promoter linked to a selectable marker. Preferably the selectable marker is one that is detrimental to non-transformed cells, e.g., methatrexate. This strategy serves to target the expression plasmid to integrate at chromosomal positions that favor high-level gene expression. Once the expression plasmid is introduced into the target host cell population, stable cell lines are selected using appropriate concentrations of the selective agent.

Any selectable marker gene can be used. These are well known in the art and include genes that change the sensitivity of a cell to a stimulus such as a nutrient, an antibiotic, etc. Genes include those for neo, dhfr, puro, tk, multiple drug resistance (MDR), etc. neo and dhfr are particularly preferred.

Any promoter which is weakly expressed in the given cell can be used. Generally, by “weak promoter” is intended a promoter that drives expression of a coding sequence at a low level. By “low level” is intended at levels of about 1/10,000 transcripts to about 1/100,000 transcripts to about 1/500,000 transcripts. Weak promoters include inducible promoters under non-inducing conditions, as well as repressible promoters. One preferred weak promoter is the H₂L^{d} promoter.

Another aspect of the invention used to achieve high levels of expression of the gene encoding the protein of interest is the use of a strong promoter linked to that gene. Preferably, the strong promoter allows expression of the gene of interest such that the protein encoded by the gene of interest can be purified from the cells at quantities of at least 1 milligram per liter. More preferably, at 5 milligrams per liter. Even more preferably, at 10 milligrams per liter. A strong promoter typically drives expression of a coding sequence at a high level, or at about 1/10 transcripts to about 1/100 transcripts to about 1/1,000 transcripts. Strong promoters are well known in the art and include, for example, the human cytomegalovirus (CMV) promoter, SV40 promoter, lac promoter, trp promoter, trc promoter, tac promoter, MPSV, the P_{R} promoter and the P_{L} promoter of lambda phage, viral LTRs including RSV LTR, HIV-1 LTR, HTLV-1 LTR, and the like. One can also use other elements to enhance expression such as the HIV tat and TAR system.

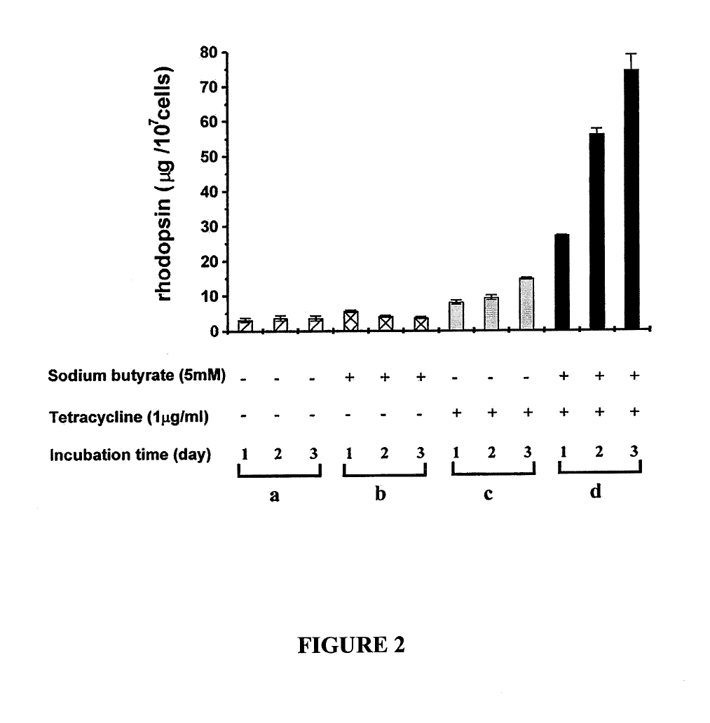

Other strong promoters include inducible promoters. The use of an inducible promoter is particularly preferred where the gene of interest encodes a protein that is toxic to the cell. Such systems include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone or rapamycin [see Miller and Vvhelan, supra at FIG. 2]. Inducible systems are available from Invitrogen, Clontech and Arlad. Systems using a repressor with the operon are preferred. Regulation of transgene expression in target cells represents a critical aspect of gene therapy. For example, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP16) to create a tetR-mammalian cell transcription activator fusion protein, tTa (tetR-VP16), with the teto-bearing minimal promoter derived from the human cytomegalovirus (hCMV) major immediate-early promoter to create a tetR-tet operator system to control gene expression in mammalian cells. Recently Yao and colleagues [F. Yao et al., Human Gene Therapy, supra] demonstrated that the tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter. One particular advantage of this tetracycline inducible switch is that it does not require the use of a tetracycline repressor-mammalian cells transactivator or repressor fusion protein, which in some instances can be toxic to cells [M. Gossen et al., Natl. Acad. Sci. USA, 89:5547-5551 (1992); P. Shockett et al., Proc. Natl. Acad. Sci. USA, 92:6522-6526 (1995)], to achieve its regulatable effects.

The effectiveness of some inducible promoters increases over time. In such cases one can enhance the effectiveness of such systems by inserting multiple repressors in tandem, e.g. TetR linked to a TetR by an IRES. Alternatively, one can wait at least 3 days before screening for the desired function. While some silencing may occur, given the large number of cells being used, preferably at least 1×10⁴, more preferably at least 1×10⁵, still more preferably at least 1×10⁶, and even more preferably at least 1×10⁷, the effect of silencing is minimal.

One can enhance expression of the gene encoding the protein of interest by known means to enhance the effectiveness of this system. In one preferred embodiment of the invention, a histone deacetylase inhibitor is used to increase expression levels of the gene of interest. Preferred histone deacetylase inhibitors include butyrates (e.g., sodium butyrate, arginine butyrate, and butyric acid); trapoxin; and trichostatin A.

In another preferred embodiment of the invention, a methylation inhibitor is used to increase expression levels of the gene of interest. A preferred methylation inhibitor is 5-azacytidine.

Expression of the gene of interest can also be enhanced, for example, using the Woodchuck Hepatitis Virus Posttranscriptional Regulatory Element (WPRE). See Loeb, V. E., et al., Human Gene Therapy 10:2295-2305-(1999); Zufferey, R., et al., J. of Virol. 73:2886-2892 (1999); Donello, J. E., et al., J. of Virol. 72:5085-5092 (1998).

Examples of polyadenylation signals useful to practice the present invention include but are not limited to human collagen I polyadenylation signal, human collagen II polyadenylation signal, and SV40 polyadenylation signal.

The protein of interest can be any protein for which it is desirable to express high levels.

In one preferred embodiment, the protein of interest is detrimental or toxic to cell growth. In this embodiment, an inducible promoter is used for the gene encoding the protein of interest, as described above.

In another preferred embodiment, the protein of interest is a glycoprotein. Glycoproteins of interest include soluble and membrane-bound proteins that are made in the endoplasmic reticulum, including those destined for transport to the Golgi apparatus, lysosomes, plasma membrane, or extracellular space are glycoproteins, as well as cytosolic proteins. Other particularly preferred glycoproteins include viral glycoproteins.

For expression of glycoproteins, it can be useful for certain purposes to generate homogenous glycosylation patterns by either eliminating glycosylation altogether or inactivating cellular enzymes to generate restricted and defined glycans.

To express a glycoprotein of interest with no glycans attached to the mature protein, the cell can be grown in the presence of a compound which eliminates glycosylation. A preferred compound is tunicamycin.

To express a glycoprotein of interest with restricted or defined glycans, the cell line can been mutated to inactivate one of the enzymes involved in the oligosaccharide processing pathway in the ER and the Golgi apparatus, including the N-glycan biosynthesis paythway. For example, the cell line can be mutated to inactivate N-acetylglucosamine transferase I by selecting for mutants resistant to a lectin including RCA(II) (ricin). Glysoproteins expressed in such a mutant cell have unelaborated GlcNAc2Man5. Other useful lectins include PHA, WGA, ConA, and LCA. The enzyme activities in this pathway sensitive to this selection include alpha-3/6 mannosidase II, GlcNAc transferase II, core alpha6-fucosyltransferase, beta3 or 4 galactose transferase, beta3 or 4 GalNAc transferase, alpha2,3 and alpha 2,6-sialyltransferase.

For example, mutants deficient in N-acetylglucosamine transferase I, a key enzyme for complex N-glycan synthesis, can be isolated by selecting for ricin resistance. by treating exponentially growing cells with a mutagen and selecting for survivors in the presence of ricin. Mutagens include ethyl methane sulfonate and ultraviolet irradiation. Ricin can be used at 1 ng/ml, 10 ng/ml, 100 ng/ ml, or 1000 ng/ml. Cells can be refed with fresh medium containing ricin until colonies form. In a preferred embodiment, the cells are HEK293S cells.

In another preferred embodiment the protein of interest is a membrane protein including membrane glycoproteins.

In a preferred embodiment, the membrane protein is a seven-transmembrane spanning G protein coupled receptor (GPCR), such as the prototypical GPCR, rhodopsin. Unlike many of the single-pass receptors, these proteins do not have enzymatic activity themselves but instead are functionally linked to signaling proteins known as G proteins. The chemokine receptor CCR5 that serves as the principal coreceptor for HIV-1 is another typical example of a G protein-coupled receptor. Other well studied members of this class include transducin, which senses light, and the acetylcholine receptor, which binds neurotransmitter at neuronal synapses.

The list of membrane proteins, including integral or transmembrane proteins, is vast. Transmembrane proteins may cross the membrane only once or over twenty times. Many transmembrane proteins associate with other transmembrane proteins to form larger complexes. Such complexes may be comprised of two identical subunits (such as homodimers) or two different protein subunits (such as heterodimers). There are examples of even larger complexes of three (sodium ion channel, Na⁺/K⁺ ATPase), four (aquaporin), five (cation channels of nicotinic receptors, anion channels of glycine receptors) or more (photoreaction center, mitochondrial respiratory chain) homologous or heterologous subunits.

Other membrane proteins of interest include transmembrane proteins that only cross the membrane once (also known as single-pass proteins). This class of proteins is particularly diverse both structurally and functionally, and includes a large number of cell surface receptors. For examples, the EGF receptor binds epidermal growth factor, which leads to activation of the receptor's tyrosine kinase activity. Other examples of single-pass transmembrane proteins include the integrins and cadherins, which function in cell-cell communication via binding to extracellular molecules.

Other membrane proteins of interest include membrane transport proteins, which fall into two general classes: a) carrier proteins, which bind the specific solute to be transported and undergo a conformational change to allow its transit, and b) channel proteins, which allow specific solutes, most often inorganic ions, to cross the membrane when they are open and form a channel. Well-studied carrier proteins include the ABC transporters (spanning the membrane 6 times), which bind solute as well as ATP and change conformation upon the hydrolysis of ATP to ADP. Many ion pumps are examples of gated carrier proteins, such as the 1 0-membrane spanning catalytic subunit of the calcium pump.

In another preferred embodiment, the membrane proteins are envelope proteins. Still more preferably, the proteins are lentiviral proteins. The lentiviral proteins can include, for example, proteins from human immunodeficiency virus (HIV), feline immunodeficiency virus (FIC), or visna virus. Preferably, the transmembrane protein is comprised of multimers of the basic unit, such as the trimeric spikes formed by HIV-1 or HIV-2 envelope proteins. The lentiviral protein is preferably from a primate lentivirus, still more preferably a human immunodeficiency virus (HIV-1), e.g. the HIV-1 gp120 or HIV-1 gp160.

Sequences of genes of interest are widely available in the literature and from computer databases such as Genbank. Thus, one can readily obtain the gene encoding a particular protein of interest. This gene can be expressed by any known means. In addition to the elements described above, other enhancer elements are known and may also be used to increase expression of the gene of interest. The codons used to synthesize the protein of interest may be optimized, converting them to codons that are preferentially used in mammalian cells. Optimal codons for expression of proteins in non-mammalian cells are also known, and can be used when the host cell is a non-mammalian cell (for example in insect cells).

The gene is then introduced into a cell for expression by known means. For example, they can include vectors, liposomes, naked DNA, adjuvant-assisted DNA, gene gun, catheters, etc. Vectors include chemical conjugates, plasmids, phage, etc. The vectors can be chromosomal, non-chromosomal or synthetic. Commercial expression vectors are well known in the art, for example pcDNA 3.1, pcDNA4 HisMax, pACH, pMT4, PND, etc.

Preferred vectors include viral vectors, fusion proteins and chemical conjugates. Retroviral vectors include Moloney murine leukemia viruses. Other vectors include pox vectors such as orthopox or avipox vectors, herpesvirus vectors such as a herpes simplex I virus (HSV) vector (Geller, A. I. et al., (1995), J. Neurochem, 64: 487; Lim, F., et al., (1995) in DNA Cloning: Mammalian Systems, D. Glover, Ed., Oxford Univ. Press, Oxford England; Geller, A. I. et al. (1993), Proc Natl. Acad. Sci.: U.S.A. 90:7603; Geller, A. I., et al., (1990) Proc Natl. Acad. Sci USA 87:1149), adenovirus vectors (LeGal LaSalle et al. (1993), Science, 259:988; Davidson, et al. (1993) Nat. Genet 3: 219; Yang, et al., (1995) J. Virol. 69: 2004) and adeno-associated virus vectors (Kaplitt, M. G., et al. (1994) Nat. Genet. 8: 148).

The particular vector chosen will depend upon the host cell used.

The introduction of the gene into the host cell can be by standard techniques, e.g. infection, transfection, transduction or transformation. Examples of modes of gene transfer are well known in the art and include e.g., naked DNA, CaPO₄ precipitation, DEAE dextran, electroporation, protoplast fusion, lipofection, cell microinjection, and viral vectors.

An antigenic tag may be inserted in the gene encoding the protein of interest to assist in its purification. Preferably, the tag is present at either the N-terminal end or the C-terminal end of the protein. The tag is preferably 6 to 15 amino acids in length, still more preferably about 6 to 9 amino acids. The tag is selected and its coding sequence inserted into the gene encoding the protein in a manner not to affect the overall conformation or function of the protein. Affinity tags are well known in the art and include immunoaffinity tags such as HA, polyoma, C9, and FLAG, metal ion affinity tags such as hexahisitidine, and enzyme-substrate tags such as GST and BCCP.

Any cell which allows high level expression of the gene of interest can be used. Preferably the cell is a mammalian or an insect cell. Particularly preferred mammalian cells include the HEK293S cell line.

The cells can be grown under conditions to optimize expression of the gene encoding the protein of interest. Conditions will vary depending on the cell line. In general, cells can be placed into any known culture medium capable of supporting cell growth, including DMEM, RPMI, HEM, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. A defined, serum-free culture medium may be preferred, as serum contains unknown components (i.e. is undefined). For example, a particularly preferable culture medium is a defined culture medium comprising a mixture of DMEM, F12, and a defined hormone and salt mixture.

The culture medium can be supplemented with a proliferation-inducing growth factor(s). As used herein, the term “growth factor” refers to a protein, peptide or other molecule having a growth, proliferative, differentiative, or trophic effect on cells. Growth factors that may be used include any trophic factor that allows cells to proliferate, including any molecule that binds to a receptor on the surface of the cell to exert a trophic, or growth-inducing effect on the cell. Preferred proliferation-inducing growth factors include Primatone RL-UF, a source of short polypeptides and amino acids, EGF, amphiregulin, acidic fibroblast growth factor (αFGF or FGF-1), basic fibroblast growth factor (bFGF or FGF-2), transforming growth factor alpha (TGFα), and combinations thereof. Growth factors are usually added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular growth factor.

In addition to proliferation-inducing growth factors, other growth factors may be added to the culture medium that influence proliferation and differentiation of the cells including NGF, platelet-derived growth factor (PDGF), thyrotropin releasing hormone (TRH), transforming growth factor betas (TGFβs), insulin-like growth factor (IGF-1) and the like.

In one embodiment the transmembrane (and optionally the cytoplasmic domain) of the membrane protein is deleted or modified so that the protein is secreted from the cell and then harvested.

Cells can be cultured in suspension or on a fixed substrate, depending on the cell type. One particularly preferred substrate is a hydrogel, such as a peptide hydrogel, as described below and in U.S. Ser. No. 09/778,200 and U.S. Ser. No. 60/305,379, which are hereby incorporated by reference. Suspension cultures are preferable for culturing large volumes of cells for protein purification. Cell suspensions can be seeded in any receptacle capable of sustaining cells, particularly culture flasks, cultures plates, or roller bottles, more particularly in small culture flasks such as 25 cm² cultures flasks.

In one preferred embodiment, cells are grown in a bioreactor to allow large-scale production. A preferred bioreactor is a Celligen Plus bioreactor equipped with pitch blade impellers (New Brunswick Scientific). Growth conditions can be optimized for the particular cell line in the bioreactor. For example, dextran sulfate can be added and calcium chloride can be omitted to reduce the tendency of the cells to aggregate in suspension cultures. Agents to induce expression of inducible promoters may be added during all phases of growth, or may only be added once the cells reach a certain density. For example, for cells expressing proteins detrimental or toxic to cell growth, gene expression may not be induced until the cells have reached the maximal density under which they express proteins at high levels.

Conditions for culturing should be close to physiological conditions. The pH of the culture medium should be close to physiological pH, preferably between pH 6-8, more preferably between about pH 7 to 7.8, with pH 7.4 being most preferred. Physiological temperatures range between about 30° C. to 40° C. Cells are preferably cultured at temperatures between about 32° C. to about 38° C., and more preferably between about 35° C. to about 37° C.

Preferably, the protein encoded by the gene of interest can be purified from the cells at quantities of at least 1 milligram per liter. More preferably, at 5 milligrams per liter. Even more preferably, at 10 milligrams per liter.

Protein purification techniques are well known in the art. The purification protocol will reflect the degree of purity required for the intended purpose. The proteins may be isolated from the bioreactor or cell culture and purified using any of a variety of conventional methods including: liquid chromatography such as normal or reversed phase, using HPLC, FPLC and the like; affinity chromatography (such as with inorganic ligands or antibodies); size exclusion chromatography; immobilized metal chelate chromatography; gel electrophoresis; and the like. One of skill in the art may select the most appropriate isolation and purification techniques without departing from the scope of this invention.

### EXAMPLES

### Example 1

Materials and Methods

Materials

Frozen bovine retinae were from W. L. Lawson (Lincoln, Nebr.). The silica column (10 μm, Econosphere, 250 mm×22 mm i.d.) used for purification of 11-cis-retinal was from Alltech Associates. Sepharose-4B was from Amersham Pharmacia. The detergents n-dodecyl-β-D-malto side (DM) and n-nonyl-β-D-gluco side (NG) were purchased from Anatrace (Maumee, Ohio). The anti-rhodopsin antibody, rho-1D4 (15), was prepared by the Cell Culture Center (Minneapolis) from a cell line provided by R. S. Molday (University of British Columbia, Vancouver). The epitope for rho-1D4, the nonapeptide corresponding to the C-terminal sequence of rhodopsin, was prepared by the Massachusetts Institute of Technology Biopolymer Laboratory. FBS, tetracycline, all-trans-retinal, and dextran sulfate (average Mᵣ 5,000) were purchased from Sigma, and sodium butyrate was from J. T. Baker (Mallinchrodt Baker, Phillipsburg, N.J.). Primatone RL-UF was a gift from Quest International (Hoffinan Estates, Ill.), calcium-free DMEM was from Atlanta Biologicals (Norcross, Ga.), and tunicamycin was from Calbiochem. Blasticidin and plasmids pCEP4 and pCDNA6-TR were from Invitrogen. Geneticin was from GIBCO/BRL. DNA oligonucleotides were purchased from Genosys (The Woodlands, Tex.). Restriction and other DNA modification enzymes were from New England Biolabs and Roche Molecular Biochemicals. The enhanced chemiluminesence detection kit was from Amersham Pharmacia.

Buffers used were as follows: buffer A (137 mM NaCl/2.7 mM KCl/1.5 mM K₂HPO₄, pH 7.2); buffer B (buffer A +1% wt/vol DM); buffer C (buffer A +0.1% wt/vol DM); buffer D {10 mM 1,3-bis[tris(hydroxymethyl)-methylamino] propane, pH 6.0+0.1% DM}; buffer E (buffer D +100 μM epitope nonapeptide); buffer F (buffer C +100 μM epitope nonapeptide); buffer G (buffer A +2% wt/vol NG); buffer H (buffer A +0.5% wt/vol NG); buffer I {10 mM 1,3-bis[tris(hydroxymethyl)-methylamino] propane, pH 6.0+0.5% NG}; buffer J (buffer I +100 μM epitope nonapeptide); and buffer K (buffer H +100 μM epitope nonapeptide).

Methods

Rod outer segments from bovine retinae were prepared as described (16). Purification of the expressed rhodopsin and its mutants was by immunoaffinity chromatography with rho-1D4-Sepharose (17). 11-cis-Retinal was prepared by illumination (>435 nm) of all-trans-retinal in ethanol (18) by using light from a slide projector equipped with a 300-W bulb. The isomers formed were separated by using isocratic HPLC with the silica column described above. The solvent mixture used was hexane containing ethyl acetate (1.3%) and isopropyl alcohol (0.1%) (vol/vol) in hexane.

Construction of the Tetracycline-Regulated Expression Plasmid, pACMV-tetO-Rho

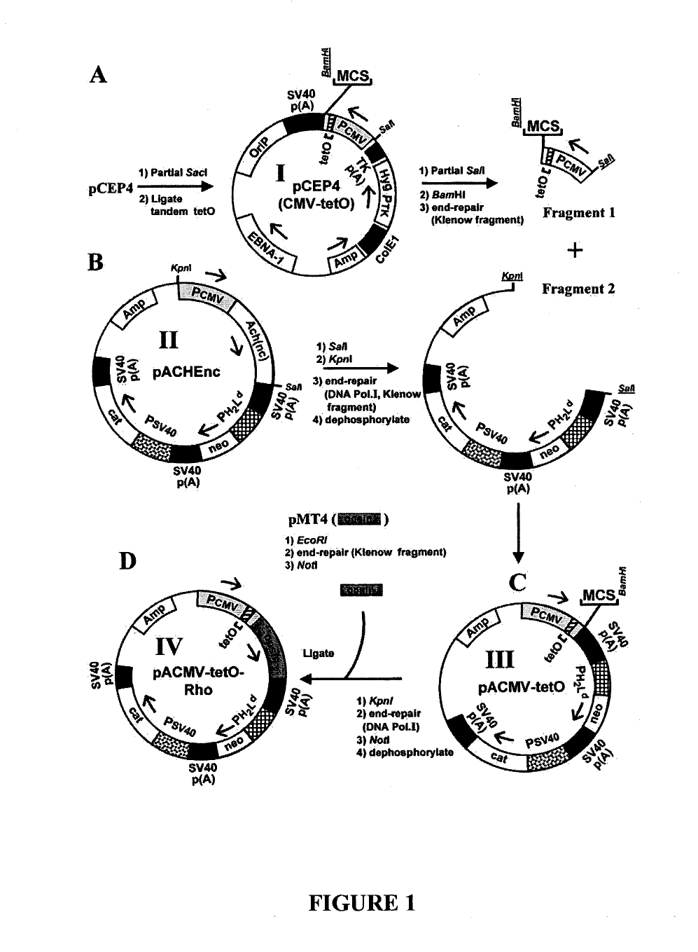

The total steps in the construction are illustrated in FIG. 1. An oligonucleotide cassette containing two tetO sequences in tandem was inserted, as described by Yao et al. (13), into the Sacd site at the 3′ end of the CMV promoter of plasmid pCEP4 (Invitrogen) to give pCEP4-(CMV-tetO) (FIG. 1AI). The DNA fragment corresponding to the CMV-tetO promoter was excised from this plasmid by partial digestion with SalI followed by complete digestion with BamHI. This DNA, designated fragment 1 (FIG. 1A), was then end-repaired with E. coli DNA polymerase I Klenow fragment and purified by using agarose gel electrophoresis. Fragment 2 (FIG. 1B) was prepared from the plasmid, pACHEne (FIG. 1B II) (19) by excising a DNA fragment containing both the entire CMV promoter and the acetylcholine esterase, Ach(nc), gene by digestion with KpnI and SalI (FIG. 1B). Fragment 2 was then end-repaired with both DNA polymerase (Pol-I) and Klenow fragment, treated with alkaline phosphatase, and purified by agarose gel electrophoresis. Blunt-end ligation of fragments 1 and 2 gave the plasmid pACMV-tetO (FIG. 1C III). The opsin gene was prepared from the plasmid pMT4 (17). The latter was digested with EcoRI, the resulting linear DNA end-repaired by using the DNA polymerase I Klenow fragment, and then digested with NotI to liberate the opsin gene, which was purified by agarose gel electrophoresis. For preparation of the plasmid, pACMV-tetO-Rho (FIG. 1D IV), the plasmid pACMV-tetO (FIG. 1C III) was digested with KpnI and the resulting linear DNA end-repaired by using DNA Pol-I, then digested with NotI, treated with alkaline phosphatase, and purified by agarose gel electrophoresis. Subsequent ligation with the opsin gene gave IV, pACMV-tetO-Rho (FIG. 1D).

Construction of stable HEK293S Cell Lines for Tetracycline-induced Opsin Gene Expression

HEK293S cells maintained as described (1) were transfected with the plasmid pCDNA6-TR (Invitrogen) carrying a gene encoding the selectable marker, blasticidin, and a gene coding for the tet operon repressor protein (TetR). The transfection method used calcium phosphate precipitation as described (1), except that the proportion of CO₂ in the incubator was reduced to 1.5%. This reduction further increased the transfection efficiency. Stably transfected cell lines resistant to blasticidin (5 μg/ml) were selected. The pool of the resulting colonies was then expanded under blasticidin selection and transfected with pACMV-tetO-Rho (FIG. 1). Cell lines stably transfected with this plasmid were selected by using Geneticin (2 mg/ml), and individual colonies appearing after 14 days were isolated and expanded as described (1). Cell lines were expanded in triplicate to 10⁷ cells in 10-cm-diameter culture dishes. Cells from one dish were stored in liquid nitrogen. Another dish was supplemented with growth medium alone, whereas the third dish was supplemented with growth medium containing both tetracycline (2 μg/ml) and sodium butyrate (5 mM). When necessary, tunicamycin was added to the growth medium 3-4 h before induction of the opsin gene. The cells were incubated for a further 48 h. For harvesting, the cells were removed from the dish, pelleted by using a clinical centrifuge, and washed with 10 ml of buffer A. The cells were repelleted by using a clinical centrifuge and resuspended in 500 μl of buffer A. 11-cis-Retinal (5 μM) was then added to the whole-cell suspension, which was mixed end-over-end by using a nutator in the dark at 4° C. for 2 h. The cells were solubilized by addition of DM to the final concentration in buffer B and incubation for 1 h. The clear supernatant was collected by centrifugation for 30 min at 50 K(TL-100 rotor) and analyzed by UV-visible absorbance spectroscopy before and after photobleaching (1 min, >495 nm light from a fiber optic).

Growth of 293S Cell Line Suspension Cultures in a Bioreactor

Preparation of the Medium

The growth vessels (2.4- and 14-liter capacity) of a Celligen Plus bioreactor were equipped with pitch blade impellers and primed for use as directed by the manufacturer (New Brunswick Scientific). The growth medium used was a calcium-free, high-glucose (4.5 g/liter) DMEM custom formulation prepared as a powder by Atlanta Biologicals. The dissolved medium was supplemented with sodium bicarbonate (3.7 g/liter) and 0.3 g/liter of Primatone RL-UF. After filtration through a 0.2-μm membrane the medium was supplemented further with the following sterile components: heat-treated FBS (10% vol/vol), penicillin G (100 units/ml), streptomycin (100 μg/ml), glutamine (292 μg/ml), dextran sulfate (300 μg/ml), and pluronic F-68 (0.1% wt/vol).

Growth of 1.1L Cultures in a 2.4L Vessel

The growth parameters were set to 37° C., pH 7, and 50% dissolved oxygen, the latter two parameters being achieved and maintained by interactive control delivery of a four-gas mixture (air, N₂, CO₂, and O₂) split between a direct sparge (up to 21 ml/min) and an overlay sparge (up to 185 ml/min). The medium was inoculated by using cells obtained by trypsinization of six confluent 15-cm-diameter dishes that had been fed the day before inoculation. Cells were stirred by using the pitch black impeller at the stirrer speed of 30-35 rpm. The cell density at inoculation was 3-5×10⁵ cells per milliliter. On day 5 after inoculation, supplements, 10 ml of 20% (wt/vol) glucose and 30 ml of 10% (wt/vol) Primatone RL-UF, were added, and expression of the opsin gene was induced on the sixth day by addition of both tetracycline (2 μg/ml) and sodium butyrate (5 mM) to the growth medium. Samples (10 ml) were removed every 24 h for determining the viable count and for analysis of protein. For determining the viable cell count, 1 ml of pelleted cells was washed with 1 ml of buffer A, repelleted, and then incubated for 1 min with 100 μl of trypsin (0.05%). The trypsinized cells were diluted to 1 ml by using the complete DMEM, diluted further if necessary, mixed with trypan blue, and counted by using a hemocytometer. Cells were harvested on day 7 or 8.

Growth of 5.5L Cultures in a 14L Vessel

The same growth medium, growth parameters, supplementation, and gene induction strategy as described above were used with the following changes. The inoculation cell density (3-5×10⁵ cells per ml) was achieved by scaling up the number of inoculum dishes to 30. For agitation the stirrer speed was set at 30 rpm and aeration was by direct sparge only starting at a flow rate of 21 ml/min on day 1 with daily increments to 374 ml/min toward the end of the incubation period

Immunoaffinity Purification

(a) Non-glycosylated Rhodopsin

Growth of the WT opsin-producing cell line was performed by using a 5.5-liter culture volume. Cells were grown as described above and treated with tunicamycin (2 μg/ml) on day 6, 4 h before induction of opsin expression. The cells were harvested 1 day later, and a portion (one-seventh) was suspended in buffer A (40 ml final volume) containing PMSF (0.1 mM) and treated with 11-cis-retinal (15 μM) for 15 h at 4° C. to constitute the pigment (20). Pigment constitution and all subsequent operations were performed in the dark or under dim red light. Cells were solubilized in buffer G [by addition of the detergent NG from a 10% (wt/vol) stock to the concentration as in buffer G] followed by end-over-end mixing for 1.5 h at 4° C. The solubilized cell extract was clarified by centrifugation (Ti-45 rotor, 35,000 rpm for 40 min), and the total amount of rhodopsin was determined by UV-visible difference spectroscopy analysis with a small portion of the solubilized material. A 50% slurry (10 ml) of Sepharose-rho-1D4 beads (capacity, 0.7 mg of rhodopsin per ml of settled beads) was added, followed by incubation for a further 1.5 h. The beads were packed into a column (1.6 cm i.d.×8.5 cm) and washed sequentially with 200 ml of buffer H and 50 ml of buffer I. Elution of rhodopsin (5-ml fractions) was performed at 22° C. by application of buffer J followed by buffer K.

(b) Triple Rhodopsin Mutant (E113Q/E134Q/M257Y)

Cell growth was performed on a 1.1-liter scale as described above, and cells were harvested 1 day after induction of opsin expression. A one-third portion of the harvested cells was suspended in 25 ml of buffer A containing PMSF (0.1 mM) and treated with 11-cis-retinal (40 μM) for 15 h in the dark. Cells were solubilized in buffer B (by addition of DM from a 20% stock to the concentration as in buffer B) for 1 h. The solubilized cell extract was clarified by centrifugation (Ti-45 rotor, 35 K, 40 min) and to this centrifugate was added 4 ml of Sepharose-rho-1 D4 beads. After 3-5 h, the beads were packed into a column (1.0 cm i.d.×7.0 cm) and the column washed with 500 ml of buffer C followed by 50 ml of buffer D. Elution of the pigment was with buffer E followed by buffer F; 2-ml fractions were collected.

Results

Tetracycline and Sodium Butyrate-Dependent Expression of WT Opsin in HEK293S Cell Lines

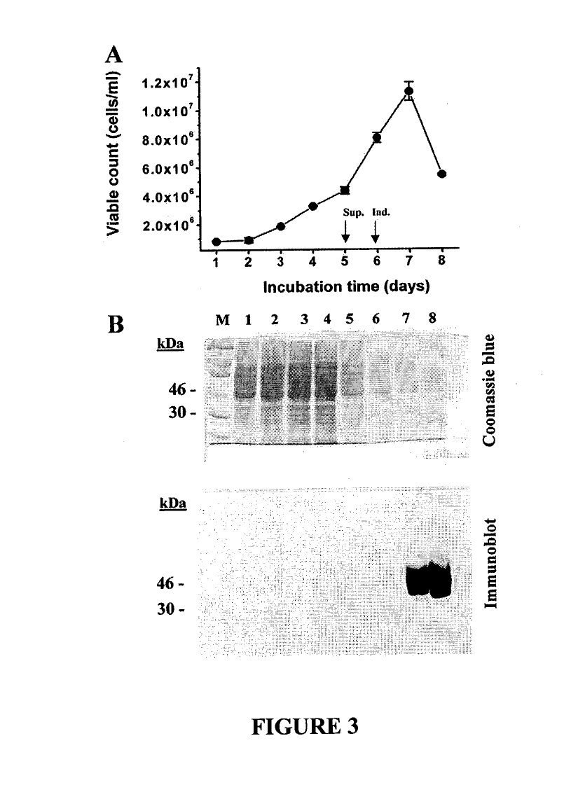

Regulatable expression of WT opsin in one stable HEK293S cell line (Materials and Methods) was probed as shown in FIG. 2. Cells were grown to near confluence in multiple 10-cm-diameter dishes. They were then supplied with fresh medium supplemented with tetracycline or sodium butyrate or both. Cells were harvested at days 1, 2, and 3, treated with 11-cis-retinal (5 μM), and after solubilization of the cells in buffer B, the yield of constituted rhodopsin was quantitated by UV-visible difference spectrum analysis (1). As analyzed in FIG. 2, uninduced cells seemed to make insignificant (<5 μg/dish) amounts of rhodopsin as did those treated only with sodium butyrate. However, the more sensitive immunodetection method (FIG. 3B) suggests that opsin gene expression in the absence of induction is even lower than that indicated in FIG. 2. Cells supplemented with tetracycline alone showed significant production of rhodopsin in a time-dependent manner. However, the presence of both tetracycline and sodium butyrate resulted in the highest level of opsin expression, the yield being up to ≈75 μg/dish after 72 h.

Inducible High Level Expression of WT Opsin in a Bioreactor

An HEK293S cell line carrying an inducible opsin gene was grown in a bioreactor by using optimal growth conditions established in Materials and Methods. Primatone RL-UF is a source of short polypeptides and amino acids and thus enriches the medium (21). Dextran sulfate (22) was added, but calcium chloride was omitted to reduce the tendency of the cells to aggregate in suspension culture. In this experiment, 1.1 liter of the medium was inoculated with cells from six confluent 15-cm dishes. An initial lag phase of 24 h was usually observed (FIG. 3A), followed by cell growth with a doubling time of ≈24-30 h. The growth medium was supplemented (Materials and Methods) on day 5, and opsin gene expression was induced on day 6 by addition of tetracycline and sodium butyrate to the medium. The cells reached a maximum density of 10⁷ cells per milliliter on day 7 (FIG. 3A). (In separate experiments cell densities of >5×10⁶ were obtained consistently.) Opsin expression was monitored in cell samples taken at 24-h intervals. Detergent-solubilized cell lysates were investigated by SDS/PAGE (10%), and the separated total proteins were visualized by Coomassie blue, whereas rhodopsin was selectively visualized by immunoblotting with rho-1D4 antibody (FIG. 3B). No opsin was detected by immunoblotting in the 6 days before induction (FIG. 3B, lanes 1-6), even when overloading of the protein occurred (FIG. 3B, Coomassie blue). Opsin production was clearly evident in the samples taken 24 and 48 h after induction (days 7 and 8). The final level of opsin at the point of harvest on day 8 was 9 mg/liter.

Expression of Non-glycosylated Opsin in the Presence of Tunicamycin

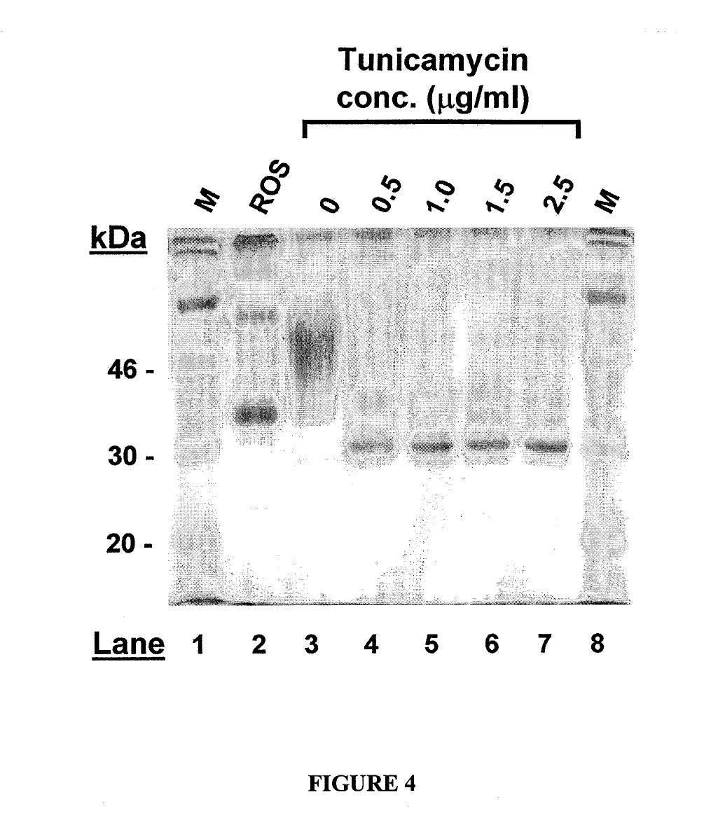

Rhodopsin purified from bovine rod outer segments migrates at an apparent Mᵣ of ≈35 kDa (FIG. 4, lane 2), whereas rhodopsin purified from tetracycline and sodium butyrate induced HEK293S cells migrates predominantly as a smear of 40-60 kDa (FIG. 4, lane 3). Induction of the opsin gene expression and inhibition of opsin glycosylation was studied in the presence of tunicamycin. As seen in lanes 4-7 (FIG. 4) tunicamycin inhibited glycosylation in a concentration-dependent (0.5-2.5 μg/ml) manner. By using tunicamycin at a level of 2 μg/ml, opsin synthesis was ≈50% ofthat from nontunicamycin-treated cells. However, the opsin made in the presence of tunicamycin was fully constituted to rhodopsin with 11-cis-retinal, as shown (23) with λₘₐₓ at 500 nm and A_{280/500} ratio of 1.6 (data not shown).

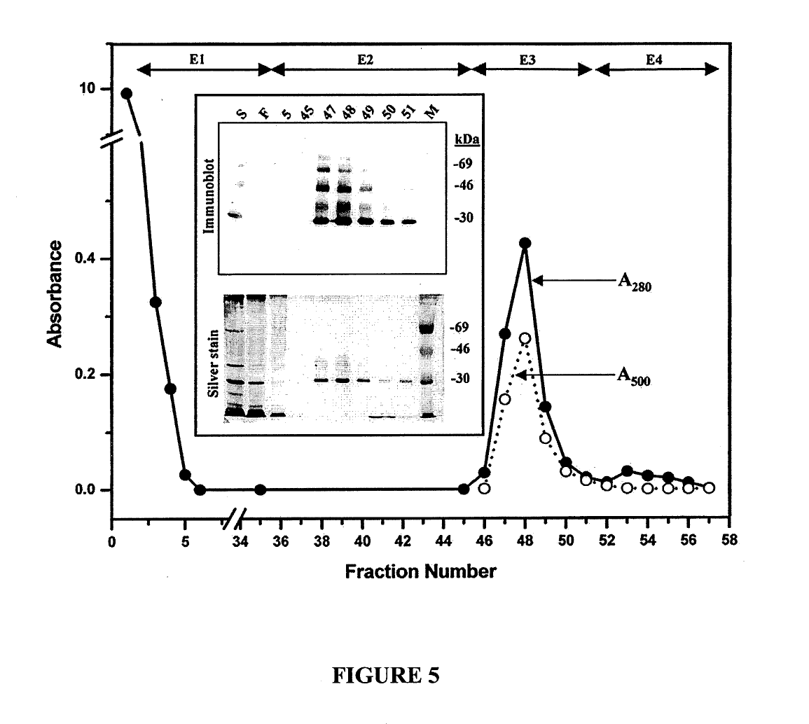

For preparation of milligram levels of nonglycosylated rhodopsin, the inducible opsin-producing HEK293S cell line was grown in 5.5-liter suspension exactly as described (Materials and Methods). On day 6 after inoculation, the cells were treated with tunicamycin (2 μg/ml), and then 4 h later with tetracycline and sodium butyrate. The cells were incubated for another 24 h before being harvested. The growth profile of the suspension culture and SDS/PAGE of samples removed daily from the bioreactor were similar to those shown in FIG. 3, except that a decline in cell viability occurred on day 7. The maximum cell density reached in this experiment was ≈5×10⁶ cells per milliliter. The cells were harvested on day 7 when the total amount of rhodopsin produced was 3 mg/liter as determined by UV-visible difference spectrum with an aliquot of 11-cis-retinal-treated cells. SDS/PAGE analysis showed that opsin was synthesized only after induction of the opsin gene (data not shown). Purification of nonglycosylated rhodopsin is shown in FIG. 5. The elution profile of nonglycosylated rhodopsin from the column is identical with that of fully glycosylated rhodopsin (1) with the majority eluting in the third column volume (fraction 48). The total amount of rhodopsin recovered from the rho-1D4-Sepharose column was 3 mg (from 3.3 mg applied with a recovery of >90%). Examination by SDS/PAGE shows the greater part of rhodopsin thus produced migrated as a relatively sharp band (FIG. 5 Inset, silver stain) with an apparent Mᵣ of ≈30 kDa. Some glycosylated rhodopsin was detected by overdeveloped immunoblot (FIG. 5 Inset). The slower migrating species with mobilities around 42 and 65 kDa presumably correspond to dimer and trimer forms, respectively, of the opsin resulting from slow denaturation.

High Level Inducible Expression of a Constitutively Active Opsin Mutant

Several attempts by using the previous methodology (1) to construct a stable HEK293S cell line that would express the opsintriple mutant, E113Q/E134Q/M257Y, were unsuccessful. This failure suggests strongly that even low-level expression of this protein is cytotoxic. This mutant opsin gene was cloned into the plasmid used for constructing inducible HEK293 S cell lines and used to transfect HEK293 S-TetR cells. Isolated Geneticin-resistant colonies were expanded and stable cell lines exhibiting inducible opsin expression were identified by rho-1D4 immunodetection after transferring soluble cell extracts to nitrocellulose. One stable cell line displaying inducible expression of this mutant was chosen for further study. Monolayer growth of cells in 10-cm-diameter culture dishes followed by induction of opsin expression by treatment with tetracycline and sodium butyrate for 24 h showed that this mutant protein was produced in the range of 30 μg of rhodopsin per 10⁷ cells (data not shown). This expression level is similar to that observed for the WT protein in the same incubation period. Incubation in the presence of tetracycline and sodium butyrate for this cell line was extremely toxic and most cells detached from the culture dish within 24 h after induction of expression.

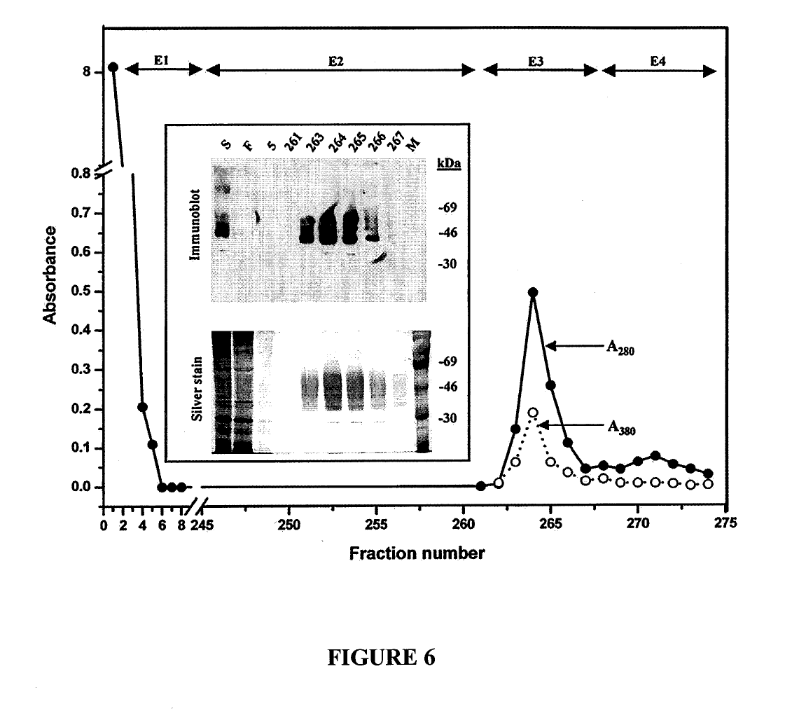

The cell line expressing this mutant opsin gene was grown in suspension culture (1.1 liter) as described in Materials and Methods. Induction of expression was restricted to 24 h. The purification of this mutant is shown in FIG. 6. The total amount of rhodopsin purified was 1.1 mg. This mutant rhodopsin combines with 11-cis-retinal to form a pigment with a λₘₐₓ of 380 nm. The glycosylation pattern (FIG. 6 Inset) is identical with WT rhodopsin produced by HEK293S cells (1) as evidenced by the migration pattern on SDS/PAGE.

In summary, in these experiments tetracycline-inducible HEK293S stable cell lines have been prepared that express high levels (up to 10 mg/liter) of WT opsin and its mutants only in response to the addition of tetracycline and sodium butyrate. The cell lines were prepared by stable transfection of HEK293 S-TetR cells with expression plasmids that contained the opsin gene downstream of a cytomegalovirus promoter containing tetO sequences as well as the neomycin resistance gene under control of the weak H₂L^{d} promoter. The inducible system is particularly suited for overcoming problems with toxicity either due to the addition of toxic compounds, for example, tunicamycin, to the growth medium or due to the expressed protein products. By optimization of cell growth conditions in a bioreactor, WT opsin, a constitutively active opsin mutant, E113Q/E134Q/M257Y, presumed to be toxic to the cells, and nonglycosylated WT opsin obtained by growth in the presence of tunicamycin have been prepared in amounts of several milligrams per liter of culture medium.

### Example 2

Materials and Methods

Materials

The materials and buffers were used as described (26) except for the following. Ricin (RCA II) was from EY Laboratories. Triton X-100 and ethyl methanesulfonate (EMS) were from Sigma. [³⁵S]Methionine (Translabel) and DMEM deficient for L-glutamine, L-cysteine, and L-cystine were from ICN. The polyclonal antibody anti-TetR was from MoBiTec (Marco Island, Fla.). ³H-labeled rainbow protein standards were from Amersham Pharmacia. The plasmid pRVSTag (29) was a gift from J. Nathans (Johns Hopkins University, Baltimore).

Arthrobacter ureafaciens sialidase (Glyko, Novato, Calif.) at 2 units/ml was used for N-glycan profiling. Trichoderma reesei α-1,2-mannosidase at 30 μg/ml was prepared from Pichia pastoris (30). APTS (8-Amino-1,3,6-pyrenetrisulfonic acid) was from Molecular Probes. The N-glycan mixture for the GnTI activity assay was prepared from bovine RNase B (Glyko).

Chemical Mutagenesis of HEK293S Cells and Isolation of Mutants Resistant to Ricin

HEK293S cell monolayers were grown in DMEM/F12 supplemented with FBS (10%), penicillin (100 units/ml), streptomycin (100 μg/ml), and glutamine (292 μg/ml). Cells in exponential growth phase were trypsinized and plated at a density of 2×10⁵ cells per ml in 70-cm² T flasks. After incubation for 20 h the growth medium was replaced with fresh medium (13 ml) containing EMS (150 μg/ml). Cells were grown in the presence of the mutagen for 20 h, washed twice with PBS, trypsinized, and then stored in three aliquots (each containing ≈8×10⁵ cells) at −70° C. One aliquot of frozen mutagenized cells was thawed and grown for 5 days to ≈70% confluence. These cells were then split 1:10 and the aliquot incubated for a further 2 days. The medium was then replaced with fresh medium containing ricin (1 ng/ml, 10 ng/ml, 100 ng/ml, or 1 μg/ml). Cells were refed with fresh medium containing appropriate levels of ricin every 4 days until colonies formed.

Identification of HEK293S Cell Lines with Altered N-Glycosylation

Ricin^{R} colonies were expanded to 10-cm dishes. Cells split 1:5 were transiently transfected the next day by using 30 μg of a mixture (10:1 wt/wt) of two plasmids (pMT4 and pRSVTag, respectively) by the Ca₂PO₄ precipitation procedure (26). Plasmid pRSVTag (29) carries a gene encoding SV40 large tumor (T) antigen that promotes pMT4 replication in HEK293 S cells (29), whereas plasmid pMT4 carries the WT opsin gene (17). Transfection was performed as described (1) but after removal of the DNA mixture, cells were incubated for 48 h to allow transient expression of the opsin gene. Cells were harvested, treated with 11-cis-retinal (5 μM), and solubilized by using buffer B (26) in the dark. Rhodopsin was purified by using mAb 1D4-Sepharose immunoaffinity chromatography (1, 17). Purified rhodopsin was subjected to SDS/PAGE (10%) and the bands were visualized by silver stain. The N-glycans inrhodopsin expressed transiently in the ricin-resistant cell lines were also analyzed by N-glycan-profiling experiments (see below).

GnTI Activity Assays: Preparation of HEK293S Cell Lysates

HEK293S and HEK293S GnTI⁻ cells were grown in 15-cm dishes.

On reaching confluence, cells were harvested and washed twice with 10 ml each of PBS buffer, and whole-cell pellets were frozen in liquid nitrogen in aliquots of ≈3×10⁷ cells. Frozen cell pellets were resuspended by addition of 200 μl of PBS containing 1% Triton X-100 and protease inhibitors (without EDTA; Roche Molecular Biochemicals). The cells were lysed by four cycles of freeze (liquid N₂)/thaw (0° C.). Insoluble material was removed by centrifugation (14,000×g, 10 min at 4° C.). Protein concentration was determined by using the bicinchonic acid procedure (9).

GnTI Activity Assays: Preparation of APTS-labeled Man₅GlcNAc₂

Bovine RNaseB N-glycans [Man₍₅₋₉₎GlcNAc₂] (30 nmol) were treated with 50 mM APTS (1,000-fold excess) in a reaction mixture (60 μl) containing citric acid (0.6 M), NaCNBH₃ (0.5 M), and DMSO (50% vol/vol). Incubation was for 4 h at 55° C. followed by 16 h at 37° C. The reaction was quenched by addition of 5 vol of water. Excess APTS was removed by Sephadex G-10 gel filtration chromatography over a 25 cm×1 cm i.d. column. Elution was performed with water at a flow rate of 0.4 ml/min. Fractions (1.2 ml) were monitored by using fluorescence spectroscopy (λₑₘ=520 nm). The fractions containing the APTS-glycan conjugate were pooled. Water was removed by evaporation (SpeedVac) and the residual APTS-labeled N-glycans were digested for 16 h by using 3 milliunits/ml of α-1,2-mannosidase in a reaction containing 20 mM sodium acetate (pH 5.0). The mixture was then desalted by gel-filtration chromatography with a 4-ml Sephadex G-10 spin column. Mannosidase was removed by precipitation by addition of methanol to 60% (vol/vol). The supernatant was then recovered and evaporated to dryness (SpeedVac). The quantity of recovered Man₅GlcNAc₂-APTS was estimated by spectrophotometry at 454 nm, assuming a molar extinction coefficient equal to the one of unconjugated APTS (ε=18,000 M⁻¹ cm⁻¹), as no molar extinction coefficient for glycan-conjugated APTS is available.

GnTI Assay Conditions

The reaction mixture (6 μl final volume) contained 50 mM Mes (pH 6.9), complete EDTA-free protease inhibitor mixture at the concentration specified by the manufacturer, 250 μM swainsonine (class II mannosidase inhibitor), 5 mM MnCl₂, 5 mM MgCl₂, ≈3 nmol Man₅GlcNAc₂-APTS, and 30 nmol UDP-GlcNAc. The reaction was initiated by addition of 1 μl of cell extract (≈25 μg of protein), and incubation was at 37° C. Aliquots (0.5 μl) of the reaction mixture were removed at different time points and the reaction was terminated by dilution (125-fold) in ice-cold water. Samples were further diluted 10-fold with water and a 1-μl aliquot was mixed with 1 μl of formamide and 0.5 μl of rhodamine-labeled oligonucleotide internal standard (Genescan 500, Applied Biosystems). Samples were applied to a polyacrylamide sequencing gel and electrophoresis was as described in the following section.

Analysis of N-Glycan in Rhodopsin

The procedure has been described in detail (32). Briefly, purified rhodopsin samples (5 μg) were bound to a poly(vinylidene difluoride) membrane in a microtiter plate format (Millipore). The N-glycans were released from the bound protein by treatment with PNGaseF and then labeled with APTS. Excess APTS was removed by gel filtration with Sephadex G-10. The labeled N-glycan mixture was supplemented with Genescan 500 rhodamine-labeled internal standard. The samples were then subjected to PAGE by using an Applied Biosystems 377A gel-based DNA-sequencer. The applied voltage was 3,500 V, and the gel temperature was maintained at 23° C. by using an external cooling bath. All exoglycosidase digestions were performed in 10 μl of 20 mM NaAc, pH 5. Data analysis was performed by using the GENESCAN 3.1 software (Applied Biosystems). A rhodamine-oligonucleotide standard used in all lanes on the same gel was used for alignment with the lane containing an APTS-labeled malto-oligosaccharide standard. The peaks corresponding to the rhodamine-labeled internal standards for alignment are omitted from the data shown.

Construction of Tetracycline-Inducible Cell Lines

The HEK293S GnTI⁻ cell line was maintained as described (1, 26), and cell growth was in the absence of ricin. Cells were transfected with plasmid pCDNA6-TR and blasticidin-resistant colonies were obtained as described (26). Individual blasticidin-resistant colonies were expanded and screened for tetR expression by analyzing solubilized cell extracts by SDS/PAGE followed by immunodetection with an anti-TetR polyclonal antibody. One anti-TetR immunopositive cell line was chosen and expanded for use in all subsequent experiments. This cell line was transfected with pACMVtetO-rho or pACMVtetO-rho (E113Q/E134Q/M257Y) (26). The HEK293S (GnTI⁻) TetR cell line was found to be hypersensitive to Geneticin, and this drug was used at 200-250 μg/ml during the selection process. After 7 days, Geneticin-resistant cells were collected by trypsinization. Purified clones were isolated from this pool of cells by limiting dilution cloning, for which it was necessary to use medium supplemented (30%) with conditioned medium (see below). Cell lines exhibiting tetracycline-inducible opsin expression were identified by immunodetection.

Conditioned medium for limited dilution cloning was prepared as follows. WT HEK293S cells were grown to 90% confluence in dishes. The medium was replaced with new medium and incubation was continued for a further 24 h. This conditioned medium was then collected and filtered (0.2-μm membrane) before storage at −20° C.

[³⁵S]Methionine Pulse-Chase in Examination of Recombinant hodopsin Maturation in Stable Cell HEK293S Cell Lines

HEK293S stable cell lines containing the inducible opsin gene were grown to 90% confluence in medium containing Geneticin (200 μg/ml) by using 10-cm dishes. The growth medium was then replaced with fresh medium containing tetracycline (2 μg/ml) and sodium butyrate (5 mM) without Geneticin. The next day the cells were grown for 1 h in the same DMEM induction medium but without methionine and cysteine and supplemented with 10% FBS that had been dialyzed with a 1-kDa exclusion membrane. The medium was then replaced with the same medium (5 ml) containing [³⁵S]methionine (100 μCi Translabel; 1 Ci=37 GBq). After incubation for 40 min (pulse time) the medium was replaced with DMEM containing unlabeled methionine (60 μg/ml) and supplemented with nondialyzed FBS but no tetracycline or sodium butyrate. After further incubations for specified time intervals, cells were pelleted and frozen immediately. Cells were solubilized in buffer B (1%) and opsin was purified by immunoaffinity chromatography. Purified opsin samples were examined by SDS/PAGE (10%) followed by autoradiography. The gels were soaked in Fluoro-Hance (Research Products International, Mount Prospect, Ill.) for 30 min and dried. Autoradiography was by exposure to Biomax film for 48 h.

Growth of 293S Cell Line Suspension Cultures in a Bioreactor

HEK293S GnTI⁻ stable cell lines harboring the inducible opsin genes were expanded in medium containing Geneticin (G418) (200 μg/ml). Cells from six 15-cm dishes were used to inoculate 1.1 liters of medium in a bioreactor (26).

Results

Construction of a Ricin^{R} HEK293S Cell Line Performing Limited N-Glycosylation

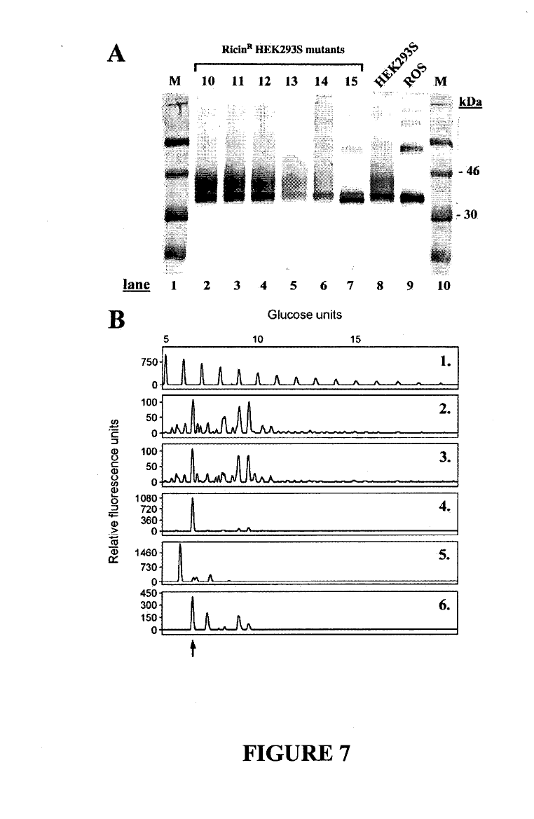

HEK293S cells growing exponentially were treated with the mutagen EMS and then incubated in the presence of ricin as described in Materials and Methods. After incubation for 2-3 weeks, ricin^{R} colonies formed in growth media containing ricin at concentrations of 1 or 10 ng/ml. No colonies were detected in dishes containing the higher concentrations, 100 ng/ml and 1 μg/ml, of ricin. The frequency of ricin^{R} colony formation at 10 ng/ml ricin concentration was 1-2 times per 10-cm dish, whereas the frequency of ricin^{R} colony formation at 1 ng/ml ricin concentration was 10-20 times higher. Isolated ricin^{R} HEK293S cell colonies were collected by using cloning rings and expanded in the absence of ricin. On reaching confluence in 10-cm dishes, ricin^{R} HEK293S cell lines were transiently transfected (Materials and Methods) by using a plasmid containing the WT opsin gene. Rhodopsin was prepared from 14 ricin^{R} cell lines obtained in the presence of 1 ng/ml ricin and one ricin^{R} cell line obtained in medium containing 10 ng/ml ricin. Purified rhodopsin was analyzed by SDS/PAGE followed by visualization by silver stain (FIG. 7A). Rhodopsin prepared from transiently transfected WT HEK293S cells (FIG. 7A, lane 8) migrated at about 34 kDa with a trailing smear. This SDS/PAGE pattern is characteristic of rhodopsin purified from transiently transfected COS-1 cells (17). The SDS/PAGE migration pattern of rhodopsin prepared from all of the HEK293 S ricin^{R} cell lines obtained in the presence of 1 ng/ml ricin (FIG. 7A, lanes 2-6) was similar to that prepared from the WT HEK293S cell line (lane 8). However, rhodopsin prepared from the single HEK293S ricin^{R} cell line obtained in the presence of 10 ng/ml ricin (FIG. 7A, lane 7) migrated as a slightly faster doublet without a trailing smear. This mobility resembles that of rhodopsin prepared from bovine rod outer segments (ROS) (FIG. 7A, lane 9).

Analysis of N-Glycans Prepared from Rhodopsin Mutants by Transient Transfection of Ricin^{R} HEK293S Cell Lines

N-glycans were released from rhodopsin mutants by treatment with PNGaseF, labeled with the fluorescent dye, APTS (Materials and Methods), and separated by PAGE. The results are shown in FIG. 7B. Profile 1 shows the mobilities of malto-oligosaccharide molecular weight standards. Rhodopsin purified from the WT HEK293S cell line (FIG. 7B, profile 2) carries heterogeneous N-glycans as indicated by numerous peaks that correspond to N-glycans of various size and monosaccharide content.

Rhodopsin prepared from all other HEK293S ricin^{R} cell lines similarly obtained displayed a very similar N-glycan profile as WT HEK293S rhodopsin (data not shown). An example of N-glycan from rhodopsin prepared from ricin^{R} cell lines obtained by using 1 ng/ml ricin concentration is shown in FIG. 7B, profile 3. Profile 4 is analysis of rhodopsin N-glycan prepared from the HEK293S ricin^{R} cell line obtained at 10 ng/ml ricin concentration. In this case, one N-glycan predominates and the single peak corresponds to 7 monosaccharide units. This peak has the same mobility as that of the Man₅GlcNAc₂ peak in the N-glycan mixture prepared from RNase B (FIG. 7B, profile 6). The N-glycan profile of ROS rhodopsin is shown in lane 5.

HEK293S Ricin^{R}-Resistant Mutant Has Undetectable GnTI Activity

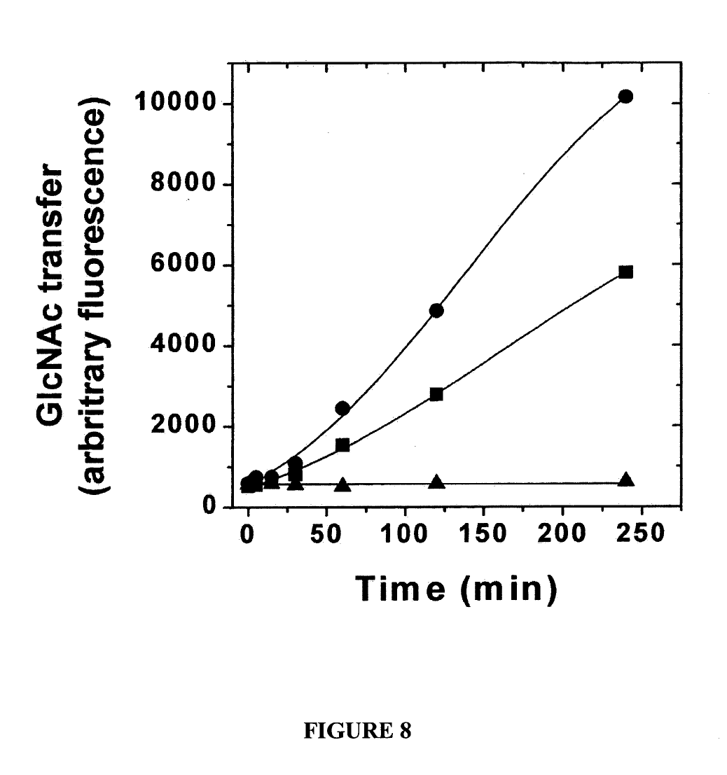

GnTI activity was measured by monitoring the transfer of GlcNAc from the donor (UDP-GlcNAc) to a fluorescently tagged acceptor (Man₅GlcNAc₂-APTS). The product (GlcNAcMan₅GlcNAc₂-APTS) was separated from the acceptor by PAGE as described in Materials and Methods. GnTI activity assays were performed by using Triton X-100-solubilized cell extracts (33) prepared from WT HEK293S cells or the HEK293S ricin^{R} mutant described in the preceding section. The results are shown in FIG. 8. The WT HEK293 S cell extract contains GnTI activity that catalyzes conversion of Man₅GlcNAc₂-APTS to GlcNAcMan₅GlcNAc₂-APTS in a time-dependent manner. No GnTI⁻ activity could be detected in the HEK293S ricin^{R} mutant. Combination of equal parts of the two cell extracts results in a GnTI activity that is approximately half of the WT value. This result demonstrates that the solubilized cell extract prepared from the HEK293S ricin mutant does not contain an inhibitor of GnTI. Thus, the HEK293S ricin^{R} mutant is completely deficient for GnTI activity, and this defect most likely arises from mutations in both GnTI alleles. The HEK293S ricin^{R} mutant deficient in GnTI activity will be hereafter referred to as HEK293S GnTI⁻.

Construction of an HEK293S GnTI⁻ Stable Cell Line for Tetracycline-Inducible Opsin Expression

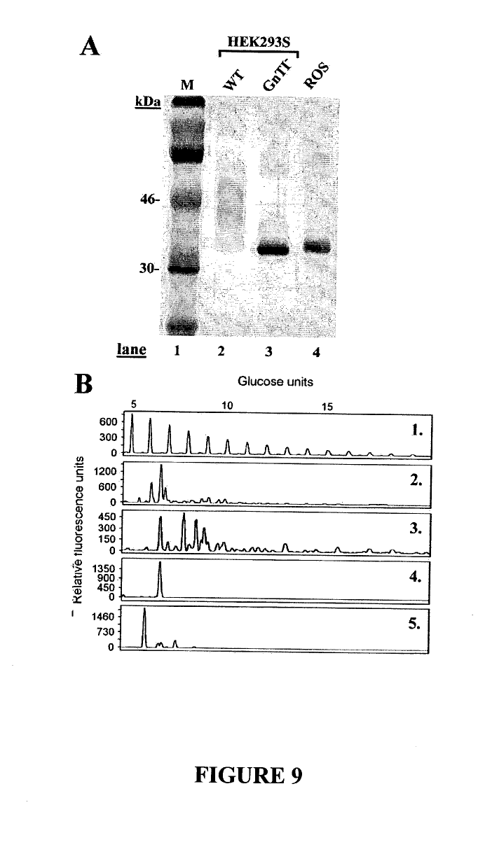

Construction of an HEK293S GnTI⁻ cell line expressing stably the tetR gene was done by transfection with plasmid pCDNA6-TR as described in Materials and Methods (26). An HEK293S GnTI⁻ cell line expressing tetR at high levels was identified. This cell line was expanded and then transfected with plasmid pACMVtetO-rho (Materials and Methods). Stable cell lines were derived from colonies arising from single cells by using limiting dilution cloning. One cell line displaying high levels of tetracycline-inducible WT opsin gene expression was identified and used in all subsequent experiments. Rhodopsin as expressed by this cell line was analyzed by SDS/PAGE and visualized by silver stain (FIG. 9A). Rhodopsin prepared after inducible opsin gene expression by using the WT HEK293S cell line and ROS rhodopsin served as markers for comparison. Rhodopsin prepared from the WT HEK293S cell line migrated as a smear, whereas rhodopsin prepared from the HEK293 S GnTI⁻ stable cell line migrated predominantly as a single species (FIG. 9A, lanes 2 and 3). The protein from the GnTI⁻ cell line migrates slightly faster than ROS rhodopsin (FIG. 9A, lane 4).

The N-glycans in the rhodopsin samples above were then analyzed. As seen in profiles 2 and 3, rhodopsin made by the WT HEK293S stable cell line has extremely heterogeneous glycans, which is further demonstrated after removal of sialic acid residues by treatment with sialidase (profile 3). In contrast, rhodopsin prepared from the HEK293S-GnTI⁻-inducible cell line gives a sharp band indicating a single glycan species (FIG. 3B, lane 4). The mobility of this species corresponds to Man₅GlcNAc₂. The N-glycan in bovine ROS rhodopsin carries mainly GlcNAc₂Man₃GlcNAc (34) (FIG. 9B, lane 5). The mobility of this glycan relative to that from HEK293S-GnTI⁻ corresponds to one less sugar unit.

Analysis of Rhodopsin N-Glycan Maturation in HEK293S Stable Cell Lines by Pulse-Chase

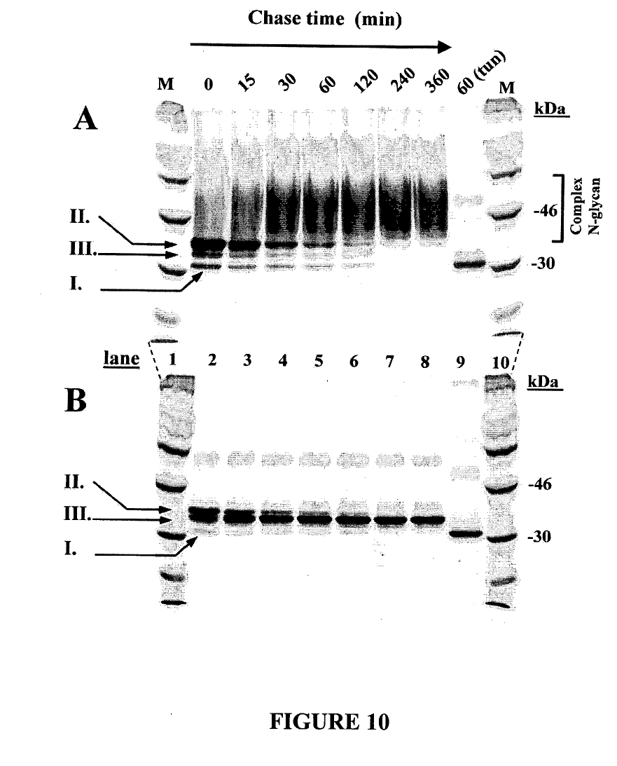

WT HEK293S and HEK293S-GnTI⁻-inducible cell lines were treated with tetracycline and sodium butyrate to induce opsin gene expression and then incubated in the medium containing [³⁵S]methionine (Materials and Methods) for 40 min. Cells were then incubated in the presence of unlabeled methionine for specified times (FIG. 10) and the opsins were purified (Materials and Methods). Opsin made by WT HEK293S cells (FIG. 10A) appears first (0- and 15-min chase) as three distinct species (FIG. 10A, lanes 2 and 3) with partial conversion to the complex form as seen by the increasing smear. The three initial protein bands presumably are nonglycosylated (species I), a major species (species II) containing the initial N-glycan, GlcNAc₃Man₅GlcNAc₂, and species III of intermediate size, presumably a trimming intermediate. Increasing chase times (FIG. 10A, lanes 4-8) show conversion of the species above to the complex form (40- to 60-kDa smear), the process being complete within 4 h. In a separate experiment, shown in FIG. 10A, lane 9, opsin was prepared from a tunicamycin-treated cell line. This nonglycosylated opsin corresponded to species I described above. As seen in FIG. 10B, lane 2, the HEK293SGnTI⁻ opsin initially formed the same pattern as described above for WT HEK293S cells, namely species I-III, but with no trailing smear. Species II is converted to species III, which then accumulates. Probably, conversion of species II to species III represents trimming of the Man₉GlcNAc₂ form to the Man₅GlcNAc₂ form.

Large-Scale Growth of the HEK293S-GnTI⁻ Cell Line for High-Level Inducible Expression of WT Rhodopsin

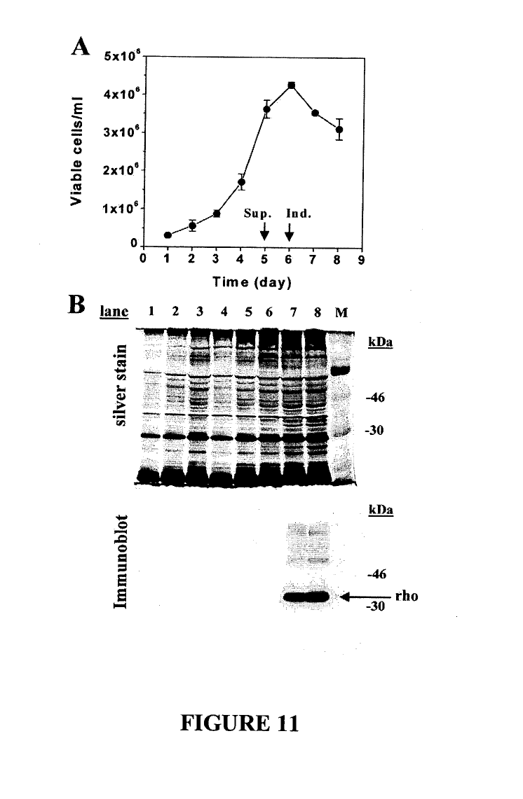

The HEK293S GnTI cell line expressing inducibly the wild-type opsin gene was grown in suspension by using a 1.1-liter bioreactor culture. The growth profile is shown in FIG. 11A. The maximum viable cell count reached on day 6 is 4×10⁶ cells per ml. Samples were collected throughout the growth curve and analyzed by SDS/PAGE (10%). Proteins were detected by silver stain (FIG. 11B Upper), whereas opsin was specifically detected by immunodetection by using mAb rho-1D4 (FIG. 11B Lower). Opsin was only detected after addition of tetracycline and sodium butyrate to the growth medium (FIG. 11B Lower, lanes 7 and 8) and migrates predominantly as a sharp band. Slower-migrating species presumably correspond to the opsin dimer and trimer. After harvest of cells and constitution with 11-cis-retinal to rhodopsin, the expression level of the opsin was calculated to be 6 mg/liter, comparable with the expression level of opsin in the HEK293S tetracycline-inducible cells (26).

Purification and Characterization of Rhodopsin Containing Man₅GlcNAc₂ N-Glycan

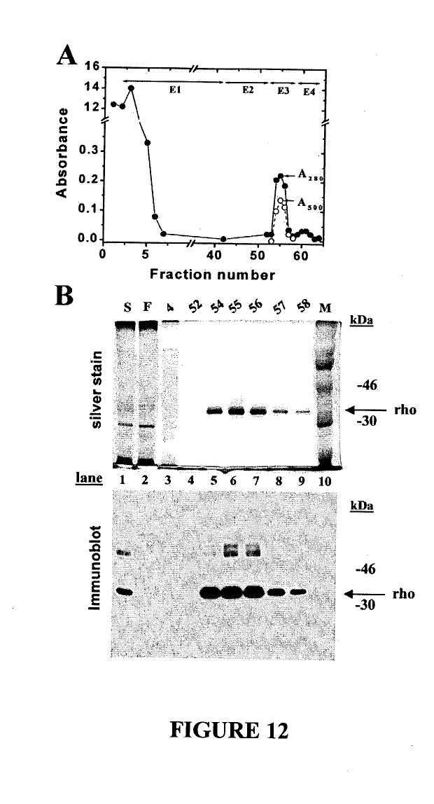

One-third portion of the harvested cells from the bioreactor run above was treated with 11-cis-retinal (17, 20) to measure rhodopsin concentration. The cells were solubilized by using nonylglucoside (2%) as in Materials and Methods (26). The supernatant from the cell extract was treated with Sepharose-rho-1D4 and after loading the beads into a column, elutions were performed, the profile being shown in FIG. 12A. Rhodopsin eluted in elution volumes 2, 3, and 4 in salt-free buffer containing the epitope peptide, E3. A small amount of A₂₈₀ absorbing material eluted in high salt shown in FIG. 12A. Analysis of the eluted proteins by SDS gel is shown in FIG. 12B. Rhodopsin migrated mainly as a single species and the A₂₈₀/A₅₀₀ nm ratio of fraction 3 was 1.6, corresponding to that of pure rhodopsin. Recovery of rhodopsin was 1.5 mg from about 2 mg (75%) of rhodopsin present in the solubilized extract.

Purification of the Rhodopsin Mutant, E113Q/E134Q/M257Y, Containing Only the Man₅GlcNAc₂ N-Glycan

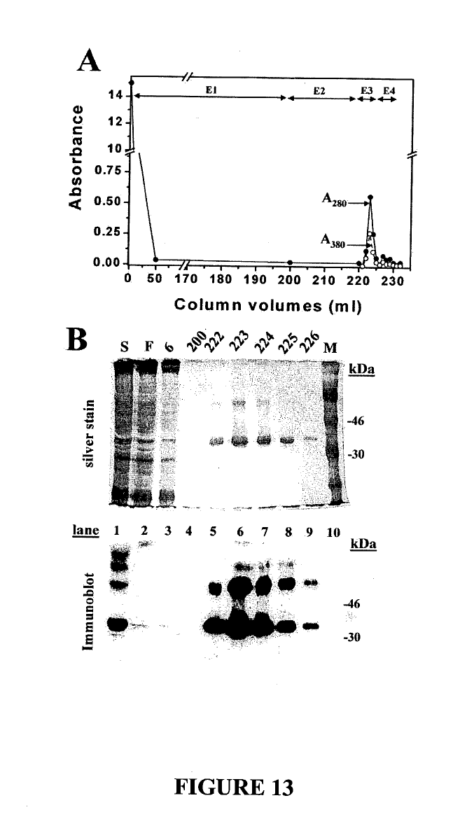

An HEK293S GnTI⁻ cell line was constructed for tetracycline-inducible expression of the rhodopsin mutant, E113Q/E134Q/M257Y. This cell line was grown in a 1.1-liter bioreactor culture, and opsin expression was induced by addition of tetracycline and sodium butyrate (26). One-third portion of the cells was treated with 11-cis-retinal and the cells solubilized by using 1% DM. The rhodopsin mutant protein was purified by rho-1D4-Sepharose chromatography (FIG. 13A). The successive elutions used are described in the FIG. 13 legend. The total yield of rhodopsin obtained was close to 1 mg. SDS/PAGE followed by silver staining and immunoblotting of rhodopsin fractions demonstrated that N-glycosylation was uniform (FIG. 13B).

In summary, these results show that an HEK293S cell line resistant to ricin was prepared by mutagenesis by using ethyl methanesulfonate. This cell line lacks N-acetylglucosaminyltransferase I (GnTI) activity, and consequently is unable to synthesize complex N-glycans. The tetracycline-inducible opsin expression system was assembled into this GnTI⁻ HEK293S cell line. Stable cell lines were isolated that gave tetracycline/sodium butyrate-inducible expression of the WT opsin gene at levels comparable with those observed in the parent tetracycline-inducible HEK293S cell line. Analysis of the N-glycan in rhodopsin expressed by the HEK293S GnTI⁻ stable cell line showed it to be Man₅GlcNAc₂. In a larger-scale expression experiment (1.1 liter) a WT opsin production level of 6 mg/liter was obtained. Further, the toxic constitutively active rhodopsin mutant, E113Q/E134Q/M257Y, previously shown to require inducible expression, has now been expressed in an HEK293S GNTI⁻-inducible cell line at levels comparable with those obtained with WT rhodopsin.

References

1. Reeves, P. J., Thurmond, R. L. & Khorana, H. G. (1996) Proc. Natl. Acad. Sci. USA 93, 11487-11492.

2. Reeves, P. J., Kein-Seetharaman, J., Getmanova, E. V., Eilers, M., Loewen, M. C., Smith, S. O. & Khorana, H. G. (1999) Biochem. Soc. Trans. 27, 950-955.

3. Eilers, M., Reeves, P. J., Ying, W., Khorana, H. G. & Smith, S. O. (1999) Proc. Natl. Acad. Sci. USA 96, 487-492.

4. Klein-Seetharaman, J., Getmanova, E. V., Loewen, M. C., Reeves, P. J. & Khorana, H. G. (1999) Proc. Natl. Acad. Sci. USA 96, 13744-13749.

5. Klein-Seetharaman, J., Reeves, P. J., Loewen, M. C., Getmanova, E. V., Chung, J., Schwalbe, H., Wright, P. E. & Khorana, H. G. (2002) Proc. Natl. Acad. Sci. USA 99, 3452-3457.

6. Loewen, M. C., Kein-Seetharaman, J., Getmanova, E. V., Reeves, P. J., Schwalbe, H. & Khorana, H. G. (2001) Proc. Natl. Acad. Sci. USA 98, 4888-4892.

7. Bruel, C., Cha, K., Reeves, P. J., Getmanova, E. & Khorana, H. G. (2000) Proc. Natl. Acad. Sci. USA 97, 3004-3009.

8. Mieschendahl, M., Petri, T. & Hanggi, U. (1986) Biotechnology 4, 802-808.

9. Remaut, E., Tsao, H. & Fiers, W. (1983) Gene 22, 103-113.

10. Kamik, S. S. , Nassal, M. , Doi, T. , Jay, E. , Sgaramella, V. & Khorana, H. G. (1987) J. Biol. Chem. 262, 9255-9263.

11. Gossen, M. & Bujard, H. (1992) Proc. Natl. Acad. Sci. USA 89, 5547-5551.

12. Fussenegger, M. (2001) Biotechnol. Prog. 17, 1-51.

13. Yao, F., Svensjo, T., Winkler, T., Lu, M., Eriksson, C. & Eriksson, E. (1998) Hum. Gene Ther. 9, 1939-1950.

14. Niu, L. , Kim, J.-M. & Khorana, H. G. (2002) Proc. Natl. Acad. Sci. USA 99, 13409-13412.

15. Molday, R. S. & MacKenzie, D. (1983) Biochemistry 22, 653-660.

16. Papermaster, D. S. (1982) Methods Enzymol. 81, 240-246.

17. Oprian, D. D., Molday, R. S., Kaufman, R. J. & Khorana, H. G. (1987) Proc. Natl. Acad. Sci. USA 84, 8874-8878.

18. Knowles, A. and Priestly, A. (1978) Vision Research 18, 115-116.

Velan, B., Kronman, C., Ordentlich, A., Flashner, Y., Leitner, M., Cohen, S. & Shafferman, A. (1993) Biochem. J. 296, 649-656.

19. Velan, B., Kronman, C., Ordentlich, A., Flashner, Y. , Leitner, M. , Cohen, S. & Shafferman, A. (1993) Biochem. J. 296, 649-656.

20. Reeves, P. J., Hwa, J. and Khorana H. G. (1999) Proc. Natl. Acad. Sci. USA 96,1927-1931.

21. Schlaeger, E-J. (1996) J. Immun. Meth. 194, 191-199.

22. Dee, K. U., Shuler, M. L. & Wood, H. A. (1997) Biotech. And Bioeng. 54, 191-205.

23. Kaushal, S., Ridge, K. D. and Khorana, H. G. (1994) Proc Natl Acad Sci U S A. 91(9):4024-8.

24. Palmero, D. P., Degraaf, M. E., Marotti, K. R., Rehberg, E. & Post, L. E. (1991) J. Biotechnol. 19, 35-47.

25. Reeves, P. J. , Callewaert, N., Contreras, R. & Khorana, H. G. (2002) Proc. Natl. Acad. Sci. USA 99, 13419-13424.

26. Reeves, P. J., Kim, J.-M. & Khorana, H. G. (2002) Proc. Natl. Acad. Sci. USA 99, 13413-13418.

27. Stanley, P., Sallustio, S., Kragg, S. S. & Dunn, B. (1990) Somat. Cell Mol. Genet. 16,211-223.

28. Stanley, P. & Chaney, W. (1985) Mol. Cell Biol. 5, 1204-1211.

29. Nathans, J. (1990) Biochemistry 29, 9746-9752.

30. Maras, M., Callewaert, N., Piens, K., Claeyssens, M., Martinet, W., Dewaele, S., Contreras, H., Dewerte, I., Penttila, M. & Contreras, R. (2000) J. Biotechnol. 77, 255-263.

31. Smith, P. K., Krohn, R. I., Hermanson, G. T. , Mallia, A. K., Gartner, F. H., Provenzano, M. D., Fujimoto, E. K., Goeke, N. M., Olson, B. J. & Klenk, D. C. (1985) Anal. Biochem. 150, 76-85.

32. Callewaert, N., Geysens, S., Molemans, F. & Contreras R. (2001) Glycobiology 11,275-281.

33. Vischer, P. & Hughes, R. C. (1981) Eur. J. Biochem. 117, 275-284.

34. Fukuda, M. N., Papermaster, D. S. & Hargrave, P. A. (1982) Methods Enzymol. 81, 214-223.

35. Kaushal, S., Ridge, K. D. & Khorana, H. G. (1994) Proc. Natl. Acad. Sci. USA 91, 4024-4028.

Landau, E. M. & Rosenbusch, J. P. (1996) Proc. Natl. Acad. Sci. USA 93, 14532-14535.

Palczewski, K., Kumasaka, T., Hori, T., Behnke, C. A., Motoshima, H., Fox, B. A., Le Trong, I., Teller, D. C., Okada, T., Stenkamp, R. E., Yamamoto, M. & Miyano, M. (2000) Science 289, 739-745.

Okada, T., Le Trong, I., Fox, B. A., Behnke, C. A., Stenkamp, R. E. & Palczewski, K. (2000) J. Struct. Biol. 130, 73-80.

Faham, S. & Bowie, J. U. (2002) J. Mol. Biol. 316, 1-6.

Luecke, H., Schobert, B., Richter, H-T., Cartailler, J-P. & Lanyi, J. K. (1999) J. Mol. Biol. 291, 899-911.

Luecke, H., Schobert, B., Lanyi, J. K., Spudich, E. N. & Spudich, J. L. (2001) Science 293, 1499-1503.

Chaney, W. & Stanley, P. (1986) J. Biol. Chem. 261, 10551-10557.

Chen, W., Unligil, U. M., Rini, J. M. & Stanley, P. (2001) Biochemistry 40, 8765-8772.

Mertens, N., Remaut, E. & Fiers, W. (1995) Biotechnology 13, 175-179.

Dunn, R. J., Hackett, N. R., McCoy, J. M., Chao, B. H., Kimura, K. & Khorana, H. G. (1987) J. Biol. Chem. 262, 9246-9254.

Stanley, P., Raju, T. S. & Bhaumik, M. (1996) Glycobiology 6, 695-9.

Oelmann, S., Stanley, P. & Gerardy-Schahn, R. (2001) J Biol Chem 276, 26291-300.

Lee, J., Sundaram, S., Shaper, N. L., Raju, T. S. & Stanley, P. (2001) J Biol Chem 276, 13924-34.

Zhang, A., Potvin, B., Zaiman, A., Chen, W., Kumar, R., Phillips, L. & Stanley, P. (1999) J Biol Chem 274, 10439-50.

Papac, D. I., Briggs, J. B., Chin, E. T. & Jones, A. J. (1998) Glycobiology 8, 445-54.

Duffin, K. L., Lange, G. W., Welply, J. K., Flonnan, R., O'Brien, P. J., Dell, A., Reason, A. J., Morris, H. R. & Fliesler, S. J. (1993) Glycobiology 3, 365-80.

All references described herein are incorporated herein by reference.

We claim:

1. An HEK293 cell line transformed by a vector system containing a selectable marker operably linked to a weak promoter and a gene of interest operably linked to a strong promoter.

2. The cell line of claim 1, wherein the strong promoter operably linked to the gene of interest is an inducible promoter.

3. The cell line of claim 1, wherein the cell line expresses at least 10 mg of the protein encoded by the gene of interest.

4. The cell line of claim 3, wherein the inducible promoter is selected from the group consisting of a tet regulated promoter and a lac regulated promoter.

5. The cell line of claim 4, wherein the tet regulated promoter is a CMV promoter containing two tet operator sequences, and the vector system is expressed in a host cell which expresses tet repressor.

6. The cell line of claim 1, wherein the strong promoter is selected from the group consisting of the human cytomegalovirus promoter, SV40 promoter, lac promoter, trp promoter, tre promoter, MPSV, and viral LTRs.

7. The cell line of claim 1, wherein the weak promoter is a repressible promoter or an inducible promoter when the host cells are grown under non-inducing conditions for that inducible promoter.

8. The cell line of claim 1, wherein the weak promoter is the H₂L^{d} promoter.

9. The cell line of claim 1, wherein expression of the gene of interest in a host cell under protein-production conditions is detrimental to the host cell.

10. The cell line of claim 9, wherein the gene of interest is operably linked to an inducible promoter, and the protein-production conditions comprise inducing conditions for the inducible promoter, and the protein expressed by the gene of interest is detrimental to the host cell.

11. The cell line of claim 1, wherein the protein-production conditions comprise addition of an agent which is detrimental to the host cell.

12. The cell line of claim 11, wherein the agent which is detrimental to the host cell is an agent which inhibits the glycosylation pathway.

13. The cell line of claim 12, wherein the agent which inhibits the glycosylation pathway is tunicamycin.

14. The cell line of claim 1, wherein the gene of interest encodes a protein selected from the group consisting of a glycoprotein, a toxic gene product, a membrane protein, a humanized protein, an antibody, a G-protein coupled receptor, and a kinase.

15. The cell line of claim 14, wherein the gene of interest encodes a G-protein coupled receptor selected from the group consisting of opsin and rhodopsin.

16. The cell line of claim 1, wherein the selectable marker is encoded by a gene selected from the group consisting of neo, dhfr, puro, tk, and MDR.

17. The cell line of claim 1, wherein the HEK293 cell line generates uniform glycosylation pattern on glycoproteins.

18. The cell line of claim 17, wherein the HEK293 cell line is a ricin resistant cell line that is defective in GnTi activity.

19. A method of producing high levels of a protein of interest in a host cell line, comprising:
(a) introducing a vector system into a host cell line, wherein the vector system comprises a selectable marker operably linked to a weak promoter and a gene of interest operably linked to a strong promoter;
(b) selecting stable transformants of said host cell line by isolating those transformants which express the selectable marker at a sufficient level to allow their survival when grown in the presence of a selecting agent; and
(c) culturing said transformants under growth conditions which allow expression of the gene of interest to produce the protein of interest.

20. The method of claim 19, wherein the growth conditions comprise addition of a histone deacetylase inhibitor to the culture medium to increase expression levels of the gene of interest.

21. The method of claim 20, wherein the histone deacetylase inhibitor is selected from the group consisting of sodium butyrate, arginine butyrate, butyric acid, trapoxin, and trichostatin A.

22. The method of claim 20, wherein the histone deacetylase inhibitor is sodium butyrate.

23. The method of claim 19, wherein the protein of interest is a glycoprotein.

24. The method of claim 23, wherein the growth conditions comprise addition of an agent which inhibits the glycosylation pathway to the culture medium, to generate a protein of interest that contains a uniform glycosylation pattern.

25. The method of claim 23, wherein the host cell line generates uniform glycosylation pattern on glycoproteins.

26. The method of claim 25, wherein the host cell line is a ricin resistant cell line that is defective in GnTi activity.

27. The method of claim 26, wherein the host cell line is an HEK293 cell line.

28. The method of claim 19, wherein the protein of interest is expressed at a high level such that it can be purified from the host cell line at a quantity selected from the group consisting of at least 1 milligram per liter, at least 5 milligrams per liter, and at least 10 milligrams per liter.

29. The method of claim 19, wherein expression of the gene of interest in a host cell under protein-production conditions is detrimental to the host cell.

30. The method of claim 29, wherein the growth conditions comprise growing a culture of the transformants to near-maximal density before switching the culture to protein-production conditions.

31. The method of claim 30, wherein the gene of interest is operably linked to an inducible promoter, and the protein-production conditions comprise inducing conditions for the inducible promoter, and the protein expressed by the gene of interest is detrimental to the host cell.

32. The method of claim 19, wherein the protein-production conditions comprise addition of an agent which is detrimental to the host cell.

33. The method of claim 32, wherein the agent which is detrimental to the host cell is an agent which inhibits the glycosylation pathway.

34. The cell line of claim 33, wherein the agent which inhibits the glycosylation pathway is tunicamycin.

35. A vector system for high level protein expression, wherein the vector system comprises a selectable marker operably linked to a weak promoter and a gene of interest operably linked to a strong promoter.
